(19) United States
(12) Patent Application Publication
(10) Pub. No.: US 2007/0148191 A1
(43) Pub. Date: Jun. 28, 2007

(54) Use of ligands of specific antigen for the diagnosis and treatment of solid tumours and bone-related cancerous diseases

(76) Inventors: Veit Krenn, Berlin (DE); Raphaela Gunther, Berlin (DE); Christine Sers, Berlin (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/582,697

(22) PCT Filed: Oct. 8, 2004
(86) PCT No.: PCT/EP04/11302

§ 371 Date: Jan. 4, 2007

(30) Foreign Application Priority Data
Dec. 19, 2003 (EP) 03029464.9

(51) Int. Cl. A61K 39/00

(52) U.S. Cl. 424/277.1

(57) ABSTRACT

The present invention relates to the use of ligands of specific antigens for the diagnosis and treatment of solid tumours and bone-related cancerous diseases, and in particular to the use of ligands for CDw52 for the preparation of a medicament for the treatment of CDw52-expressing solid tumours, such as bone tumours.

The present invention relates to the use of ligands of specific antigens for the diagnosis and treatment of solid tumours and bone-related cancerous diseases, and in particular to the use of ligands for CDw52 for the preparation of a medicament for the treatment of CDw52-expressing solid tumours, such as bone tumours.

For the purposes of the present invention, all references as cited herein are incorporated by reference in their entireties.

### DESCRIPTION

Human CDw52 is a glycosylphosphatidylinositol (GPI)-anchored antigen expressed on the all lymphocytes as well as within the male genital tract, where it can be produced by epithelial cells of the distal epididymis and duct deferens. The physiological role of CDw52 on lymphocytes is unclear, although antibody directed to CDw52, CAMPATH-1, is capable of complement activation and it can be clinically useful to treat lympho-proliferative disorders and to deplete lymphocytes in bone marrow transplants (Domagala A, Kurpisz M. CDw52 antigen—a review. Med Sci Monit. 2001 March-April; 7(2):325-31).

Presently, CDw52-specific antibodies are used in B-cell chronic lymphocytic leukaemia (B-CLL) (Moreton P, Hillmen P. Alemtuzumab therapy in B-cell lymphoproliferative disorders. Semin Oncol. 2003 August; 30(4):493-501; Lundin J, et al. Phase II trial of subcutaneous anti-CD52 monoclonal antibody alemtuzumab (Campath-1H) as first-line treatment for patients with B-cell chronic lymphocytic leukaemia (B-CLL). Blood. 2002 Aug. 1; 100(3):768-73.). The efficiency of the therapy correlates with the quantity of the CDw52 expression (D'Arena G, et al. Quantitative evaluation of CD52 expression in B-cell chronic lymphocytic leukaemia. Leuk Lymphoma. 2003 July; 44(7): 1255-7.). The CDw52 antibody is furthermore used in the therapy of transplantation rejection (Hale G, et al. CD52 antibodies for prevention of graft-versus-host disease and graft rejection following transplantation of allogeneic peripheral blood stem cells. Bone Marrow Transplant. 2000 July; 26(1):69-76.) and, most recently, in Behcet's disease (Lockwood C M, Hale G, Waldman H. Jayne D R. Remission induction in Behcet's disease following lymphocyte depletion by the anti-CD52 antibody CAMPATH 1-H. Rheumatology (Oxford). 2003 Aug. 29).

Alemtuzumab (Campath-1H, Ilex Pharmaceuticals, San Antonio, IX, USA) is a humanised monoclonal antibody that recognises the CDw52 antigen expressed on malignant and normal B lymphocytes. It has come to be used therapeutically in B-cell malignancies. Responses are seen in non-Hodgkin's lymphoma (NHL), and alemtuzumab can induce molecular remissions in relapsed chronic lymphocytic leukaemia (CLL), even when refractory to purine analogues. Most studies reveal the responses to be superior in the absence of bulky disease. Infusion-related side effects such as rigors, hypotension, and nausea are reduced by using the subcutaneous route of administration. Infectious complications are the most important toxicity seen and are related to the depletion of normal lymphocytes. The clinical efficacy in combination with both fludarabine and rituximab is under investigation.

Alemtuzumab is an unconjugated, humanised, monoclonal antibody directed against the cell surface antigen CDw52 on lymphocytes and monocytes. In noncomparative phase I/II studies in patients with B-cell chronic lymphocytic leukaemia (B-CLL) relapsed after or refractory to alkylating agents and fludarabine, intravenous (IV) administration of alemtuzumab 30 mg/day three times weekly for up to 12 weeks was associated with overall objective response (OR) rates of 21-59% (Frampton J E, Wagstaff A J. Alemtuzumab. Drugs. 2003; 63(12):1229-43; discussion 1245-6).

The biological effect of ligands of CDw52 seems to be mediated by an unusual lytic effect on the target cells (Kirk A D, et al. Results from a human renal allograft tolerance trial evaluating the humanized CD52-specific monoclonal antibody alemtuzumab (CAMPATH-1H). Transplantation. 2003 Jul. 15; 76(1):120-9).

CA 2,060,384 describes a method of treatment of a human or animal subject suffering from T-cell mediated inflammation of the joints, such as rheumatoid arthritis, which comprises administering to said subject an effective amount of an antibody recognising the CDw52 antigen. Preferably the antibody is CAMPATH-1H administered in an amount of 1 to about 100 mg per day for a period between 1 and 30 days.

WO 93/01296 describes a recombinant vaccinia virus capable of expressing on infection of a suitable cell the light chain and/or the heavy chain of an antibody and the use of such a virus in the production of recombinant antibodies. The antibody may be a chimeric or a humanised (CDR-grafted) antibody for example a humanised antibody against the CDw52 antigen.

WO 93/08837 describes a stabilised immunoglobulin composition comprising at least one immunoglobulin together with a stabilising amount of a chelator of copper ions such as EDTA or citrate. Preferably the immunoglobulin is an antibody, for example a recombinant CDR-grafted antibody against the CDw52 antigen, most preferably CAMPATH-1H.

CH 681305 describes a purified preparation of an anti-CDw52 antibody which exhibits on size exclusion chromatography: a single peak under non-reducing conditions and two major peaks under reducing conditions. Also a process of purifying an anti-CDw52 antibody, formulations containing such a purified preparation and uses thereof are described.

U.S. Pat. No. 5,786,176 and WO 94/02604 describe recombinant cell lines, in particular mammalian cell lines, capable of expressing recombinant CDw52 antigen or an antigenic fragment thereof. The cell lines can be used for the production of recombinant CDw52 antigen and in assaying for anti-CDw52 antibody in a sample.

U.S. Pat. No. 6,120,766 and WO 93/10817 describe the use of an antibody recognising the CDw52 antigen in the treatment of multiple sclerosis. Preferably, the antibody is the humanised antibody in the humanised antibody CAMPATH-1H.

WO 93/07899 describes the use of an antibody recognising the CDw52 antigen for the treatment of T-cell mediated inflammation of the joints, in particular for the treatment of rheumatoid arthritis. Preferably the antibody is the humanised antibody CAMPATH-1H which may be used together with another immunosuppressive antibody or an immunosuppressive agent such as a steroid.

Claudins are components of tight junctions (TJ) between epithelial cells (Furuse M, F. K., Hiiragi T, Fujimoto K, Tsukita S. 1998a. Claudin-1 and -2: novel integral membrane proteins localizing at tight junctions with no sequence similarity to occludin. J Cell Biol, 141: 1539-1550, Furuse M, S. H., Fujimoto K, Tsukita S. 1998b. A single gene product, claudin-1 or -2, reconstitutes tight junction strands and recruits occludin in fibroblasts. J Cell Biol, 143: 391-401, Sonoda N, F. M., Sasaki H, Yonemura S, Katahira J, Horiguchi Y, Tsukita S. 1999. Clostridium perfringens enterotoxin fragment removes specific claudins from tight junction strands: Evidence for direct involvement of claudins in tight junction barrier. J Cell Biol, 147: 195-204) and they are directly involved in paracellular sealing as well as in membrane domain differentiation (barrier and fence function; Anderson J M, V. I. C. 1995. Tight junctions and the molecular basis for regulation of paracellular permeability. Am J Physiol, 269: G467-G475, Gumbiner, B. M. 1993. Breaking through the tight junction barrier. J Cell Biol, 123(6 Pt 2): 1631-1633). They also have been shown to bind to the TJ protein ZO-1 through their carboxyl terminus. ZO-1 is believed to interact with several proteins involved in cell signalling and transcriptional regulation (Balda M S, M. K. 2000. The tight junction protein ZO-1 and an interacting transcription factor regulate ErbB-2 expression. Embo J, 19. 2024-2033. 2000, Mitic L L, V. I. C., Anderson J M. 2000. Molecular physiology and pathophysiology of tight junctions I. Tight junction structure and function: lessons from mutant animals and proteins. Am J Physiol Gastrointesat Liver Physiol, 279(G250-G254)); these studies suggest that claudins may play an indirect role in cell signalling and transcriptional regulatory elements. Another group has shown that Claudin 4 is overexpressed in pancreatic and prostate cancer as well as in ovarian carcinoma (Michl P, Buchholz M, Rolke M, Kunsch S, Lohr M, McClane B, Tsukita S, Leder G, Adler G, Gress T M. Claudin-4: a new target for pancreatic cancer treatment using Clostridium perfringens enterotoxin. Gastroenterology. 2001 September; 121(3):678-84). Since Claudin 4 has been described as a receptor for the cytotoxic Clostridium perfringens enterotoxin (CPE), Michl et al. investigated the effect of CPE on pancreatic cancer cells. They suggest that targeting Claudin 4-expressing tumours with CPE represents a promising new treatment modality for pancreatic cancer and other solid tumours. Based on its role as a TJ protein, it is furthermore suggested that overexpression of Claudin 4 in ovarian tumour cells may enhance and stabilise tumour cell connections and could contribute to increase growth at secondary site (Hibbs, K., Skubitz, K. M., Pambuccian, S. E., Casey, R. C., Burleson, K. M., Oegema, T. R., Jr., Thiele, J. J., Grindle, S. M., Bliss, R. L., & Skubitz, A. P. 2004. Differential gene expression in ovarian carcinoma: identification of potential biomarkers. Am J Pathol, 165(2): 397-414).

MME (CD10) is a type II integral zinc-dependent membrane protein with neutral endopeptidase activity. The function of MME is through to involve hydrolysis of polypeptides such as inflammatory mediators in the extracellular milieu. It regulates biological activities of peptide substrates by reducing the local concentrations available for signal transduction and receptor binding. Beside haematopoietic tissue, various benign and malignant nonhaematopoietic tissues express MME (Metzgar R S, B. M., Jones N H, Dowell B L. 1981. Distribution of common acute lymphoblastic leukemia antigen in nonhematopoietic tissues. J Exp Med, 154: 1249-1254, Sato Y, I. F., Hinoda Y, Ohe Y, Nakagawa N, Ueda R, Yachi A, Imai K. 1996. Expression of CD10/neutral endopeptidase in normal and malignant tissues of the human stomach and colon. J. Gastroenterol., 31: 12-17). MME may also play a role in perpetuation of homeostasis, neoplastic transformation and tumour progression. The exact role of MME is currently not known, but recent studies suggest that MME expression in malignant cells significantly correlates with higher apoptotic index. Interestingly, in carcinomas of the lung and kidney, expression of MME is downregulated in comparison with benign tissues. Fukushima et al. (Fukushima T, Sumazaki R, Koike K, Tsuchida M, Matsui A, Nakauchi H. Multicolor flow-cytometric, morphologic, and clonogenic analysis of marrow CD10-positive cells in children with leukemia in remission or nonmalignant diseases. J Pediatr Hematol Oncol. 1998 May-June; 20(3):222-8.) describe the analysis of MME in cells of the marrow. A similar analysis is described by Longacre et al. (Longacre T A, Foucar K, Crago S, Chen I M, Griffith B, Dressler L, McConnell T S, Duncan M, Gribble J. Hematogones: a multiparameter analysis of bone marrow precursor cells. Blood. 1989 February; 73(2):543-52. Marie et al. (Marie J P, Choquet C, Perrot J Y, Thevenin D, Pillier C, Boucheix C, Zittoun R. In vitro depletion of clonogenic cells in adult acute lymphoblastic leukaemia with a CD10 (anti-cALLA) monoclonal antibody. Eur J Cancer Clin Oncol. 1987 August; 23(8):1181-7.) describe the targeting of MME-positive cells with a monoclonal antibody in leukaemia.

Protein kinases mediate most of the signal transduction in eukaryotic cells, regulating cellular metabolism, transcription, cell cycle progression, cytoskelletal rearrangement, apoptosis and differentiation. The receptor tyrosine kinases (RTK) have well-established roles in both normal physiology and oncogenesis. Ephrin molecules are overexpressed in a wide range of neoplasms and additional evidence suggests that overexpression is an important component of carciogenic processes. The Ephrin A1 gene (efna1) has well-established potential it act as a classical oncogene (Maru Y, H. H., Takaku F. 1990. Overexpression confers an oncogenic potential upon the eph gene. Oncogene, 5: 445-447). It was found as an immediate early response gene of endothelium induced by TNF-α that possesses angiogenic and endothelial cell chemoattractant activity (Holzmann L B, M. R., Dixit V M. 1990. A novel immediate-early response gene of endothelium is induced by cytokines and encodes a secreted protein. Mol. Cell. Biol., 10(5830), Pandey A, S. H., Marks R M, Polyerini P J, Dixit V M. 1995. Role of B61, the ligand for the Eck receptor tyrosine kinase, in TNF-alpha-induced angiogenesis. Science, 268: 567-569). The Ephrin-pathway may plays a potential role in the progression of GCT. As such, the Ephrin ligands and receptors represent targets for chemotherapeutic drug development.

FGFR3 (fibroblast like growth factor receptor) is a FGF transmembrane receptor. It contains an extracellular ligand-binding domain, a transmembrane domain and a cytoplasmatic domain containing a split tyrosine kinase domain. FGF receptors are activated by FGF ligands; the binding leads to receptor dimerization and autophosphorylation of tyrosine residues in the cytoplasmatic domain. This stimulates intrinsic tyrosine kinase activity. The combination provides a mechanism to both recruit and phosphorylate molecules that transmit FGFR3 signals. The pathway downstream of the receptor are not well defined; implicated in the pathway are MAP (mitogen-activated protein) kinase, STAT1 (signal transduction and activation of transcription proteins) and PLC (phospholipase C). FGFR3 have been associated with cranio-synostosais syndromes, hypochondroplasia, achondroplasia and thanatophoric dysplasia. The effect of FGFR3 signalling on bone growth is inhibitory because it is expressed in proliferating chondrocytes. Further, a role of FGFR3 in myeloma is described (Pollett J B, Zhu Y X, Gandhi S, Bali M, Masih-Khan E, Li Z, Wen X Y, Stewart A K. RU486-inducible retrovirus-mediated caspase-3 overexpression is cytotoxic to bcl-xL-expressing myeloma cells in vitro and in vivo. Mol. Ther. 2003 August; 8(2):230-7; Martinelli G, Tosi P, Ottaviani E, Soverini S, Tura S. Molecular therapy for multiple myeloma. Haematologica. 2001 September; 86(9):908-17.

Autocrine motility factor (AMF) is a tumour secreted cytokine that regulates cell motility and metastasis (Liotta, L. A., Mandler, R., Murano, G., Katz, D. A., Gordon, R. K., Chiang, P. K., & Schiffmann, E. 1986. Tumor cell autocrine motility factor. Proc Natl Acad Sci USA, 83(10): 3302-3306). AMF stimulates arbitrary and direct cell motility via its receptor AMFR. It has been shown, that increased expression of AMFR correlates with a high incidence of recurrence and a shortened survival in patients with bladder cancer and other cancer forms (Hirono, Y., Fushida, S., Yonemura, Y., Yamamoto, H., Watanabe, H., & Raz, A. 1996. Expression of autocrine motility factor receptor correlates with disease progression in human gastric cancer. Br J Cancer, 74(12): 2003-2007). Funasaka et al. (Funasaka T, Haga A, Raz A, Nagase H. Tumor autocrine motility factor induces hyperpermeability of endothelial and mesothelial cells leading to accumulation of ascites fluid. Biochem Biophys Res Commun. 2002 April 26; 293(1):192-200) describe a treatment experiment with anti-AMF antibody succeeded in the reduction of the ascites accumulation, which renders AMF a target molecule. It is suggested that AMF is one of the significant factors which relates to various pathological malignancies induced by tumour mass, and understanding of its function could benefit prognosis and treatment.

Solid tumours make up about 30% of all paediatric cancers. The most common types of solid tumours in children include brain tumours, neuroblastoma, rhabdomyosarcoma, Wilms' tumor, and osteosarcoma (see, for example, Kline N E, Sevier N. Solid tumors in children. J Pediatr Nurs. 2003 April; 18(2):96-102.). As one additional example, high grade periacetabular osteosarcomas extending to the sacro-iliac region present a difficult management problem. Functional outcome is poor and the prognosis for this group of patients is not improved by surgery (Pollock R C, et al. The swing procedure for pelvic ring reconstruction following tumour excision. Eur J Surg Oncol. 2003 February; 29(1):59-63).

The giant cell tumour of the bone (GCT) is a local osteolytic neoplasm with variable degrees of aggressiveness (Osaka S, T. M., Taira K, Sano S, Saotome K. 1997. Analysis of giant cell tumor of bone with pulmonary metastases. Clin Orthop: 253-261, Wülling M, E. C., Jesse N, Werner M, Kaiser E, Delling G. 2001. The nature of giant cell tumor of bone. J Cancer Res Clin Oncol, 127: 467-474, Zheng M H, R. P., Xu J, Huang L, Wood D J, Papadimitriou J M. 2001. The histogenesis of giant cell tumour of bone: A model of interaction between neoplastic cells and osteoclasts. Histol Histopathol, 16: 297-307). In the first report by Jaffe published in 1940 (Jaffe J L L, P. R. 1940. Giant cell tumor of bone—Its pathologic appearance, grading, supposed variants and treatment. Arch Pathol, 30: 993-1031), it was classified to be a neoplasm of the osteoclast lineage due to the characteristic histological appearance which displays a high number of osteoclast-like multinuclear giant cells. Accordingly, Jaffe defined it as an “osteoclastoma”. Later studies identified the fibroblast-like stromal cells present in GCTs as the neoplastic component however, the origin of the neoplastic stromal cells remained unknown until today (Oreffo R O, M. G., Kirchen M, Garcia C, Gallwitz W E, Chavez J, Mundy G R, Bonewald L F. 1993. Characterization of a cell line derived from a human giant cell tumor that stimulates osteoclastic bone resorption. Clin Orthop, 296: 229-241, Goldring S R, R. M., Petrison K K, Bhan A K. 1987. Human giant cell tumors of bone identification and characterization of cell types. J Clin Invest, 79(2): 483-491).

Among all primary bone tumors GCTs represent 5% (Wülling M, E. C., Nesse N, Werner M, Kaiser E, Delling G. 2002. Aktuelles zur Histiogenese des Riesenzelltumors. Pathologe, 23: 332-339), with 80% of GCTs typically have a benign course with a local recurrence rate of 20% to 50% after intralesional curretage (Ghert M A, R. M., Harrelson J M, Scully S P. 2002. Giant cell tumor in the appendicular skeleton. Clin Orthop, 400: 201-210, Masui, F. 1998. Giant cell tumor of bone: a clinicopathologoc study of prognostic factors. Pathol Int, 48(723-729), Rock, M. 1990. Curettage of giant cell tumor of bone. Factors influencing local recurrences and metastasis. Chir Organi Mov, 75: 43-46). Due to its ambiguous biological behavior, GCT was initially defined as semimalignant. However, 10% of the GCTs undergo malignant transformation at recurrence (e.g. malignant fibrous histiocytoma, fibrosarcoma or osteosarcoma) and 1% to 4% even produce pulmonary metastases despite a benign histology (Antal I, S. Z., Szendroi M. 2000. Die maligne Entartung eines Riesenzeiltumors am distalen Radiusende. Orthopäde, 29: 677-683).

In 90% of the cases, the lesions occurs in the epiphysis of long tubular bones (Delling, G. 1997. Skelettsystem. Remmele W (Hrsg) Pathologie Band 5. Springer, Berlin Heidelberg New York Tokyo: 263-382), predominantly affecting young adults after closure of the growth plate (Carrasco C H, M. J. 1989. Giant cell tumors. Orthop Clin North Am, 20: 395-405, Salzer-Kuntschik, M. 1998. Differential diagnosis of giant cell tumor of bone. Verh Dtsch Ges Pathol, 82: 154-159). GCTs are rarely seen under the age of 10 years (1.3%) and have a male:female ratio of 3:2 (Campanacci, M. 1990. Bone and soft tissue tumors. New York, Springer: 117-151, Goldenberg R, C. C., Bonfiglio M. 1970. Giant-Cell tumor of Bone: An analysis of two hundred and eighteen cases. J Bone Joint Surg, 52A: 619-664).

GCTs exhibit three histologically different cell types. In addition to the characteristic multinuclear giant cells which express the calcitonin receptor, TRAP activity and other phenotypic osteoclast markers, two mononuclear cell types are frequently detected (Goldring S R, R. M., Petrison K K, Bhan A K. 1987. Human giant cell tumors of bone identification and characterization of cell types. J Clin Invest, 79(2): 483-491). The first cell type is a spindle-shaped, fibroblast-like stromal cell whereas the second one has a round morphology and resembles CD68-positive monocytes. Cell culture experiments have established that the stromal cells are the only proliferating components of GCTs. Monocytes and the osteoclast-like giant cells have been shown to disappear after a few culture passages (Abe Y, Y. K., Nishida K, Takagi K. 1994. Giant cell tumor of bone: analysis of proliferative cells by double-labeling immuno-histochemistry with anti-proliferating cell nuclear antigen antibody and culture procedure. Nippon Seikeigeka Gakkai Zasshi, 68: 407-414). Therefore it is likely that the fibroblast-like cell population controls the formation of osteoclast-like giant cells in the neoplasms (Goldring 1987).

The histiogenesis of GCT is uncertain. Previous studies have shown that DNA content, histological grading, tumour size and the presence of pathological fractures at diagnosis are not related to recurrence rates and do not predict the biological aggressiveness of GCTs (Katz E, N. M., Okon E, Zajicek G, Robin G. 1987. Growth rate analysis of lung metastases from histologically benign giant cell tumor of bone. Cancer, 59: 1831-1836, Ladanyi M, T. F., Huvos A. 1989. Benign metastasizing giant cell tumours of bone. Cancer, 64: 1521-1526). Therefore, its necessary to search for factors related to tumour progression that may be predictive of clinical outcome in GCT patients. There are still many unanswered questions with regard to both its treatment and prognosis. Accordingly, to delineate the neoplastic process in cases of metastatic bone disease, where the primary tumour cannot be determined with conventional modalities, new tools are attractive. cDNA and oligonucleotide array technologies have made it possible to measure the relative expression levels of thousands of genes in a single experiment. The techniques are particularly powerful for the identification of genes differentially expressed between individual samples. Currently, most tumours are categorised on the basis of morphology. In addition, the identification of markers that distinguish subtypes of tumours and which may have prognostic and therapeutic implications has been achieved in recent years for several tumour types (Abdollahi, T., Robertson, N. M., Abdollahi, A., & Litwack, G. 2003. Identification of interleukin 8 as an inhibitor of tumor necrosis factor-related apoptosis-inducing ligand-induced apoptosis in the ovarian carcinoma cell line OVCAR3. Cancer Res, 63(15): 4521-4526, Van't Veer L J, D. H., van de Vijver M J, He Y D, Hart A A, Bernards R, Friend S H. 2003. Expression profiling predicts outcome in breast cancer. Breast Cancer Res, 5(1): 57-58).

Giant cell tumours typically can be found in hollow bones (joint hub) and in flat bones (pelvic belt). Therapeutically these tumours can pose particular problems in that they can be localised in areas in which they can not be removed (e.g. sacral bone, cranial skeleton). Treatment of giant-cell tumour of bone generally involves wide en bloc resection of the lesion and the surrounding bone or curettage with or without bone-grafting or the use of cement. Radiation therapy has been used for patients who cannot be operated on for medical reasons or who have a tumour that is technically difficult to resect or that cannot be resected because of its location (Chakravarti A, et al. Megavoltage radiation therapy for axial and inoperable giant-cell tumor of bone. J Bone Joint Surg Am. 1999 November; 81(11):1566-73).

For all of the above discussed bone-related cancers, alternative diagnosis and treatment options are needed. It is therefore an object of the present invention, to provide both new diagnostic and treatment options for those bone-related tumours and in particular giant cell tumours or osteosarcomas that express specific antigens, and in particular the CDw52 antigen. Other objects, features and advantages of the present invention will be set forth in the detailed description of preferred embodiments that follows, and in part will be apparent from the description or may be learned by practice of the invention. These objects and advantages of the invention will be realised and attained by the methods and compositions particularly pointed out in the written description and claims hereof.

In one major aspect of the present invention, the object of the present invention is achieved by the use of a ligand of CDw52 for the preparation of a medicament for the treatment of CDw52 expressing solid tumours. Surprisingly, it could be found that certain solid tumours and bone-related tumours and in particular giant cell tumours or osteosarcomas express the cellular antigen CDw52, which in leukaemia is already used as a target for therapy (see above). Therefore, the present invention extends this uses to cancers that are currently difficult to treat and/or can not be treated at all.

The effectiveness of inhibitors of the antigen CDw52 for solid tumours and bone-related tumours and in particular giant cell tumours or osteosarcomas is particular surprising, since CDw52 in these cells is almost exclusively present as an intracellular antigen.

One major advantage of the use according to the present invention is based on the fact that the effect of CDw52-interacting ligands on the tumour cell is independent from the speed and/or amount of cellular proliferation activity. Preferably said CDw52 expressing solid tumours are therefore selected from the group of bone tumours, in particular slowly proliferating bone tumours. Particularly preferred are CDw52 expressing solid tumours that are selected from the group of giant cell tumours, resting solid tumours (e.g. by chemotherapy), and/or osteosarcomas (although the latter do not proliferate at a slow rate).

In principle, the ligand that forms the active principle of the present invention can be selected from all CDw52-interacting ligands, as long as the ligand also leads to the CD52-mediated death of a CDw52-expressing solid tumour cell. Thus, according to a preferred aspect of the present invention, the ligand can be selected from a CDw52-specific antibody, a fragment thereof a CDw52-binding peptide and a CDw52-interacting substance.

As already mentioned above, currently CDw52 is already used as a target for therapy in leukaemia. Here, the ligand is alemtuzumab (Campath-1H, see above). The leukaemia therapy based on Alemtuzumab is well characterised, and the person of skill in the art will be readily able to amend the described therapeutical regimen in order to treat other CDw52-expressing solid tumours, such as giant cell tumours, resting solid tumours and/or osteosarcomas. Examples of the respective literature are Moreton P and Hillmen P (Alemtuzumab therapy in B-cell lymphoproliferative disorders. Semin Oncol. 2003 August; 30(4):493-501. Review.); Cao T M and Coutre S E (Management of advanced chronic lymphocytic leukemia. Curr Hematol Rep. 2003 January; 2(1):65-72. Review.); Lynn A et al. (Treatment of chronic lymphocytic leukemia with alemtuzumab: a review for nurses. Oncol Nurs Forum. 2003 July-August; 30(4):689-94. Review.); Stull D M (Targeted therapies for the treatment of leukemia Semin Oncol Nurs. 2003 May; 19(2):90-7. Review.); Dearden C E Alemtuzumab in lymphoproliferate disorders. Rev Clin Exp Hematol. 2002 December; 6(4):435-48; discussion 449-50. Review.); Frampton J E and Wagstaff A J (Alemtuzumab. Drugs. 2003; 63(12):1229-43; discussion 1245-6. Review.); Cao T M and Coutre S E (T-cell prolymphocytic leukemia: update and focus on alemtuzumab (Campath-1H). Hematology. 2003 February; 8(1):1-6), and Tallman M S (Monoclonal antibody therapies in leukemias. Semin Hematol. 2002 October; 39(4 Suppl 3):12-9) (all incorporated by reference in their entireties) and the respective literature cited therein. Furthermore, variants of alemtuzumab can also be used for the treatment according to the present invention, as they exhibit improved properties (Hutchins J T, et al. Improved biodistribution, tumor targeting, and reduced immunogenicity in mice with a gamma 4 variant of Campath-1H. Proc Natl Acad Sci USA. 1995 Dec. 19; 92(26):11980-4.). CAMPATH-1M is a rat IgM monoclonal antibody which has been used extensively in vitro for purging bone marrow harvests in order to deplete the T cell population prior to bone marrow transplantation. Marked reduction in the incidence and severity of graft-versus-host disease has been seen with this therapy. CAMPATH-1G is a rat IgG2b class-switch variant of an IgG2a antibody recognising the CDw52 antigen which has been used in vivo to achieve immunosupression in more than 100 patients undergoing organ and bone marrow transplantation, management of organ rejection and treatment of haematologic malignancies with a high level of success. However, the rapid development of an anti-rat immunoglobulin response, including the possibility of anaphylaxis, is likely to limit the use of rat monoclonal antibodies against the CDw52 antigen in humans in vivo. CAMPATH-1H is a genetically manipulated IgG antibody obtained by grafting the complementarity determining regions from CAMPATH-1G into human framework regions. The resulting “humanised” antibody is highly effective in vitro being equivalent to the rat monoclonal antibody at complement lysis and two to four times better in cell-mediated lysis of human lymphocytes. No limiting anti-globulin response is anticipated with this humanised antibody.

In another particularly preferred embodiment of the present invention, the ligand is administered systemically and/or administered locally. The local administrating has the advantage of reduced side effects by an at the same time increased effective amount of ligand that is present at the tumour site. Subcutaneous administration of alemtuzumab has been described as a key advancement in the treatment of CLL. A phase II trial of subcutaneous alemtuzumab has demonstrated an 87% response rate in 38 previously untreated patients, with a reduction in intravenous administration-related rigors, as well as the elimination of nausea, dyspnea, diarrhea, and hypotension, frequently seen following intravenous administration of alemtuzumab. Furthermore, encouraging results with the combination of alemtuzumab and fludarabine, which demonstrate eradication of malignant cells in patients who are resistant to either agent alone, were described to open the way for such combinations to produce durable responses even in refractory disease (Rai K, Hallek M. Future prospects for alemtuzumab (MabCampath). Med Oncol. 2002; 19 Suppl: S57-63.). This subcutaneous administration of alemtuzumab can be taken as an example to readily apply such route of administration for the treatment of tumours according to the present invention.

The dosage of the ligand of CDw52, preferably the anti-CDw52 antibody, preferably CAMPATH-1H, to be administered to a patient suffering from solid tumours will vary with the precise nature of the condition being treated and the recipient of the treatment. The dose will generally be in the range 1 to about 100 mg for an adult patient, usually administered daily for a period between 1 and 30 days. The preferred daily dose is 1 to 10 mg per day although in some instances larger doses of up to 40 mg per day may be used. Preferably the dosage will be applied in such a manner that the ligand is present in the medicament in concentrations that provide in vivo concentrations of said ligand in a patient to be treated of between 0.01 mg/kg/day and 1 mg/kg/day.

The ligand of CDw52, preferably the anti-CDw52 antibody, particularly CAMPATH-1H will generally be administered to the patient in the form of a pharmaceutical formulation. Such formulations preferably include, in addition to the ligand/antibody, a physiologically acceptable carrier or diluent, possibly in admixture with one or more other agents such as other antibodies or drugs, such as an antibiotic, Suitable carriers include, but are not limited to, physiological saline, phosphate buffered saline, phosphate buffered saline glucose and buffered saline. Alternatively the ligand, e.g. the antibody, may be lyophilised (freeze dried) and reconstituted for use when needed by the addition of an aqueous buffered solution as described above. Routes of administration are routinely parenteral, including intravenous, intramuscular, subcutaneous and intraperitoneal injection or delivery. The administration can be systemic and/or locally.

The ligand of CDw52, particularly the anti-CDw52 antibody, may be administered in combination with or sequentially with a safe and effective amount of other drugs, in particular other drugs conventionally used in the treatment of solid tumours, or other medicaments for the alleviation of specific symptoms of the disease. Such drug is preferably selected from the group consisting of a DNA-interactive agent, alkylating agent, antimetabolite, tubulin-interactive agent, and a hormonal agent, and is, for example, selected from the group consisting of Methotrexate, Fluorouracil, Fluorodeoxyuridin, CB3717, Azacitidine, Floxuridine, Mercapyopurine, 6-Thioguanine, Pentostatin, Cytarabine, and Fludarabine. Additional chemotherapeutic agents are well-known to the skilled artisan in the field of cancer therapy. Sequential administration of the ligand/antibody with another drug may be appropriate in some cases (see, for example, Montillo M, et al. Successful CD34+ cell mobilisation by intermediate-dose Ara-C in chronic lymphocytic leukaemia patients treated with sequential fludarabine and Campath-1H. Leukemia. 2003 Oct. 30).

In another aspect of the present invention, the present invention provides an improved method for screening for CDw52 ligands comprising the steps of: a) incubating a cell expressing CDw52 with a putative ligand, b) measuring, if a binding between CDw52 and said putative ligand occurs, c) in the case of a binding of said ligand to CDw52 is measured, measuring, if said binding between CDw52 and said identified ligand leads to a CDw52-mediated death of a CDw52-expressing solid tumour cell.

In one embodiment, these ligands can be identified based on the structural similarities of CDw52 with other proteins, such as B7-Ag, gp20 or CD24. Examples for ligands of these small glycosylphosphatidylinositol (GPI) anchored peptides are P-selectin and/or glycans as potential recognition signals (Aigner S, et al. CD24, a mucin-type glycoprotein, is a ligand for P-selectin on human tumor cells. Blood. 1997 May 1; 89(9):3385-95.; Tone M, et al. Structure and chromosomal location of mouse and human CDw52 genes. Biochim Biophys Acta 1999 Sep. 3; 1446(3):334-40.).

During the course of the assay of the method according to the invention, the ligand will initially bind or attach to CDw52. The ligand can either bind directly or indirectly to CDw52, i.e. via cofactors that can be present, such as certain ions or protein factors, that promote the attachment of the ligand to CDw52 and therefore support the function of the ligand. “Binding” can occur via a covalent or non-covalent attachment of the ligand or group of ligands to CDw52. Based on the binding properties of the screened ligands, a first pre-selection of ligands can be performed, in which a non-binding ligand is screened in a second “round” of screening using a set of co-factors. If still no binding occurs, the ligand will be classified as “non-binding” and disregarded in further screenings. Such pre-selection will be encompassed by the terms “screening”, “measuring” and/or “determining” in the general context of this invention. A ligand that shows an in-vitro modulating (e.g. lytic) action should in vivo preferably not further interact with components of the patients' or test (model) organisms' body, e.g. within the bloodstream, lung and/or heart of the patient or test organism.

In general, assays to determine a binding and biological effect of a ligand to a specific target (in this case CDw52) are well known to the person skilled in the art and can be found, for example, in U.S. Pat. Nos. 4,980,281, 5,266,464 and 5,688,655 to Housey (which are herein incorporated by reference) for phenotypic changes of cells after incubation with a screening ligand. Furthermore, U.S. Pat. No. 5,925,333 to Krieger at al. (which is herein incorporated by reference) describes methods for modulation of lipid uptake and related screening methods.

Suitable tests for showing a biological effect of a ligand for CDw52 include lytic assays that measure the release of intracellular contents (uric acid, potassium, phosphorus) into the extracellular compartment, fluorescence based assays (e.g. use of confocal fluorescent microscopy), viable cell counts, cellular proliferation assays, such as the BrdU proliferation assay or measuring of cellular calcium, and the like.

The method of screening according to the present invention can be performed in several different formats. One embodiment is a method, wherein the assay is performed in vitro. The screening assays of the present invention preferably involve the use of bone-cancer cells and other cells, as long as these cells express CDw52. How to produce such recombinant cells is well known to the skilled artisan and is further described in the respective literature.

In another embodiment of the present invention, the ligands are initially screened using an assay such as one of the assays described herein and then tested in, for example, transgenic, CDw52-expressing and/or over-expressing, animals made using standard transgenic animal technology. A technique such as embryonic stem cell technology using rats, mice or hamsters is preferred.

An additional embodiment of the present invention relates to a method wherein the assay is performed in vivo. Preferably, the assay is performed in a mouse or rat. In general, the in vivo assay will not be substantially different from the above-mentioned in vitro assay. In a general screening assay for ligands of CDw52 will be provided in that the ligand to be tested is/are administered to a mouse or a rat. Then, it will be determined, if said ligand leads to a CDw52-mediated death of a CDw52-expressing solid tumour cells compared to the absence of the ligand to be tested, wherein a difference in the death of cells identifies a ligand which leads to a CDw52-mediated death of a CDw52-expressing solid tumour cells of said mouse or rat. Of course, these assays can be performed in other non-human mammals as well.

An additional embodiment of the present invention relates to a method according to the invention, wherein said ligand is selected from a library of naturally occurring or synthetic compounds which are randomly tested for binding to CDw52. Such libraries and the methods how to build up such a library, as well as methods for using these libraries for the screening of candidate ligands are well known to the person skilled in the art and further described in the respective literature. Furthermore, some of these libraries are commercially available. The present invention contemplates high throughput screening of ligands for CDw52. The ligands as described above, and modifications of said ligands, including analogues, derivatives, fragments, active moieties, and the like, may be screened using methods and systems of the present invention.

In the context of the present invention, a “CDw52-mediated death” encompasses all biological effects within a cell that are mediated an/or initiated by a binding of a ligand of CDw52 and lead to a negative effect for the viability and/or proliferation of the cell or tissue that expresses CDw52. Without limitation, such negative effects include the lysis of the cell, apoptosis and/or inhibition of the proliferative activity of the cell. Preferably, the effect is the CDw52-mediated lysis of the cell (for references, see above).

An additional embodiment of the present invention relates to a method for the production of a pharmaceutical formulation, comprising the steps of: a) performing a method as given above, and b) formulating the identified ligand for CDw52 with pharmaceutically acceptable carriers and/or excipients. Such formulations therefore include, in addition to the ligand/antibody, a physiologically acceptable carrier or diluent, possibly in admixture with one or more other agents such as other antibodies or drugs, such as an antibiotic. Suitable carriers include, but are not limited to, physiological saline, phosphate buffered saline, phosphate buffered saline glucose and buffered saline. Alternatively the ligand, e.g. the antibody, may be lyophilised (freeze dried) and reconstituted for use when needed by the addition of an aqueous buffered solution as described above. Routes of administration are routinely parenteral, including intravenous, intramuscular, subcutaneous and intraperitoneal injection or delivery. The administration can be systemic and/or locally.

Finally, the present invention provides novel methods for the treatment of solid and particularly bone-related tumours and in particular giant cell tumours or osteosarcomas that express CDw52 by the application to a patient in need thereof of an effective amount of a ligand, in particular an inhibitor, of the antigen CDw52 in order to induce the CDw52-mediated cellular death of the cancer cell that form the cancer to be treated. The major advantages of this novel therapy are its application in areas of the body where a surgical intervention is impossible, in the case of a multifocal disease statuses, and the application in slowly proliferating cancers (such as giant cell tumours) where current chemotherapeutical approaches can not be employed or are not effective. Furthermore, the spectrum of side effects of the treatment based o CDw52 is regarded as low (Lockwood C M, Hale G, Waldman H., Jayne D R. Remission induction in Behcet's disease following lymphocyte depletion by the anti-CD52 antibody CAMPATH 1-H. Rheumatology (Oxford). 2003 Aug. 29). Finally, the follow-up use of the ligands for CDw52 after surgery (or other treatment in order to reduce the size of the tumour) is envisaged in order to further minimise the size of the residual tumour (if not fully removed) and/or to avoid relapses.

The present invention provides a method of treatment of a human subject suffering from a solid tumour that expresses the CDw52 antigen, such as certain bone-related tumours, such as osteosarcomas and giant cell tumours, which comprises administering to the said subject an effective amount of an antibody recognising the CDw52 antigen. Suitable formulations, routes of administrations and dosages are indicated above and are further laid out in the following examples. Further examples are described in, for example, Keating M J, et al. (Campath-1H treatment of T-cell prolymphocytic leukaemia in patients for whom at least one prior chemotherapy regimen has failed. J Clin Oncol. 2002 Jan. 1; 20(1):205-13.); Montillo M, et al. (Safety and efficacy of subcutaneous Campath-1H for treating residual disease in patients with chronic lymphocytic leukemia responding to fludarabine. Haematologica. 2002 July; 87(7):695-700).

The person of skill will be aware that the above described methods and assays as described for CDw52 can be easily modified in order to be analogously applied for the following GCT-specific antigens that can be applied in similar fashions in order to solve the object of the present invention. As will be also understood, the present invention also encompasses the combination of two or more of the described antigens, both in the cases of a diagnosis and/or therapy of solid tumours, in particular solid tumours of the bone, such as GCT.

Therefore, in another important aspect of the present invention, the present invention provides for the use of a ligand of a cellular marker selected from the group comprising CDw52 (CDw52), Claudin 7 (CLDN7), Ephrin A1 (EFNA1), AMFR, MME (CD10), FGFR3 for the preparation of a medicament for the treatment of solid tumours expressing at least one of said cellular markers.

Preferably, the cellular marker expressing solid tumours are selected from the group of bone tumours, in particular slowly proliferating bone tumours. More preferably, the cellular marker expressing solid tumours are selected from the group of giant cell tumours, chondrosarcomas, and osteosarcomas.

As specified herein, the ligand is preferably selected from a cellular marker-specific antibody, a fragment thereof, a cellular marker-binding peptide, and a cellular marker-interacting substance. In one embodiment the ligand is alemtuzumab (Campath-1H).

For application, the inventive ligand can be administered systemically and/or administered locally. Accordingly, the ligand can be present in the medicament in concentrations that provide in vivo concentrations of said ligand in a patient to be treated of between 0.01 mg/kg/day and 1 mg/kg/day. As specified herein, the ligand can be suitable for administration in combination with other chemotherapeutically active substances.

In yet another aspect, the ligand is for a specific treatment of mGCs, macrophage-like cells, and fibroblast-like cells of the tumour. The specific use of the ligand depends from the marker(s) that is/are chosen as targets for the treatment. The selection of the target can be performed based on the information as given herein, for example as can be taken from the tables in the examples, below.

In yet another aspect, the present invention is related to the use of a cellular marker selected from the group comprising CDw52, CDLN7, EFNA1, AMFR, MME, FGFR3 for the diagnosis of solid tumours expressing at least one of said cellular markers. Such expression analyses are well known to the person of skill and can encompass determination of mRNA, cDNA or gene products, such as the protein level and/or amount of one or more of the markers. Preferably, the cellular marker expressing solid tumours to be diagnosed are selected from the group of bone tumours, in particular slowly proliferating bone tumours. More preferably, the cellular marker expressing solid tumours are selected from the group of giant cell tumours, chondrosarcomas, and osteosarcomas. In one particular embodiment, the diagnosis comprises the distinction between mGCs, macrophage-like cells, and fibroblast-like cells of the tumour. One example of a diagnosis using a microarray is described in the accompanying examples, whereby factors are specifically up-regulated or down-regulated in certain cellular types of the tumour. Shown in the examples are genes with differential expression between primary tumour and relapse tumour with a change fold of at least 2, nevertheless, relevant changes can be between in the order of between 1.5 to 50 fold, preferably between 1.5 to 20 fold, and most preferred between 1.5 to 5 fold.

As specified herein, yet another aspect of the present invention relates to an improved method for screening for ligands of a cellular marker selected from the group comprising CDw52, CLDN7, EFNA1, AMFR, MME, FGFR3, comprising the steps of: a) incubating a cell expressing at least one marker selected from CDw52, CLDN7, EFNA1, AMFR, MME, and FGFR3 with a putative ligand, b) measuring, if a binding between at least one marker selected from CDw52, CLDN7, EFNA1, AMFR, MME, and FGFR3 and said putative ligand occurs, c) in the case of a binding of said ligand to at least one marker is measured, measuring, if said binding between said at least one marker and said identified ligand also leads to a marker-mediated death of a marker-expressing solid tumour cell.

Yet another aspect of the present invention relates to a method for the production of a pharmaceutical formulation, comprising the steps of: a) performing a method as outlines above, and b) formulating the identified ligand for said at least one marker with pharmaceutically acceptable carriers and/or excipients.

As will be understood by the person of skill, the present invention also encompasses the uses of other markers as mentioned in the tables in the examples herein. The person of skill is able to select certain combinations of markers in order to improve the efficiency of a medication or the quality of a diagnosis, in particular in order to selectively target mGCs, macrophage-like cells or fibroblast-like cells of the tumour.

In the context of the present invention, the inventors analysed the expression pattern of GCT and found new genes which were expressed differentially in GCT. Although a gene expression study of GCT has been recently reported by Randall et al. 2003 (Randall R L, W. M., Albritton K H, Coffin C M, Joyner D E. 2003. Validation of cDNA microarray analysis to distinguish tumour type ex vivo. Clin Orthop, 415S: 110-119), but the background of this study were different when compared to the present assignment. The specific aim of the analysis by Randall et al. were to assess cDNA microarray reproducibility of serial tissue preparations of tumour samples and to confirm that heterogeneity within a given clinical tumour specimen does not preclude this technique as a tool to discern tumour types. The group used a 6912-minimally redundant cDNA chip to analyse microarray data derived from ex vivo tumour samples (malignant peripheral nerve sheat tumour, GCT and fibromatosis). The focus of the present invention has been to analyse the differentially expression pattern of ex vivo GCTs that may be involved in the pathogenesis and progression of primary and relapse tumour samples. Using immunohistochemistry, the inventors were able to approve the expression pattern of 6 selected genes identified by gene expression microarray analysis. Using RT-PCR, and immunohistochemistry, the inventors were able to approve the expression pattern of all of the 6 selected genes identified by gene expression microarray analysis. Many of these genes recognised by the analyses of the inventors concerned an important biological process, such as receptor tyrosine kinases, suggesting that many of these genes be part of the neoplastic and recurrence progression. As described earlier the tumour is very heterogeneous. Three major cell types are present, the predominant multinucleated giant cells, the macrophage-like cells and the fibroblast-like cell population, whereas those represent the neoplastic components of the tumour. Macrophage-like cells and fibroblast-like cells extinguish each 25% of all cells. The most strongly expressed genes within the downregulated GCT samples covered various lymphocyte markers. It is based on the fact that relapse tumours show a much higher infiltration. 80% of GCTs have a benign course with a local rate of recurrence of 20% to 50%. The focus of the present invention has been on using gene profiles or discovers genes that may be involved in the pathogenesis of the recurrence process. The inventors now analysed five primary tumours and two relapse tumours, whereas a subset of six genes identified was validated for the gene expression patterns by RT-PCR and/or immuno-histochemistry.

Claudins are components of tight junctions (TJ) between epithelial cells (Furuse 1998a, b, Sonoda 1999) and they are directly involved in paracellular sealing as well as in membrane domain differentiation (barrier and fence function; Anderson 1995, Gumbiner 1993). They also have been shown to bind to the TJ protein ZO-1 through their carboxyl terminus. ZO-1 is believed to interact with several proteins involved in cell signalling and transcriptional regulation (Balda 2000, Mitic 2000); these studies suggest that claudins may play an indirect role in cell signalling and transcriptional regulatory elements. The present invention displays CLDN7 mRNA and protein in multinuclear giant cells as well as in macrophage-like cells and fibroblast like stromal cells both in primary and relapse in GCT for the first time. It is widely accepted that the loss of cell-to-cell adhesion in neoplastic epithelium is necessary for invasion of surrounding stromal elements and subsequent metastatic events. Other studies show, that HGF (hepatocyte growth factor) disrupts tight junction function in human breast cancer cells by effecting changes in the expression of tight junction; they conclude that regulation of tight junctions could be of fundamental importance in the prevention of metastasis of breast cancer cells. Another group has shown, that Claudin 4 (CLDN4) is overexpressed in pancreatic and prostate cancer as well as in ovarian carcinoma. Based on its role as a TJ protein they suggest that overexpression of CLDN4 in ovarian tumour cells may enhance and stabilise tumour cell connections and could contribute to increase growth at secondary site (Hibbs, K., Skubitz, K. M., Pambuccian, S. E., Casey, R. C., Burleson, K. M., Oegema, T. R., Jr., Thiele, J. J., Grindle, S. M., Bliss, R. L., & Skubit, A. P. 2004. Differential gene expression in ovarian carcinoma: identification of potential biomarkers. Am J Pathol, 165(2): 397-414). Interestingly, the present invention shows that claudin 7 on immunohistochemical analysis were down-regulated in relapse tumours compared to primary tumours. Reasons form the basis of mean value analysis for relapse tumour samples as the account of relapse tumour was higher than in primary tumour. This suggests that CLDN7 plays a role in progression of recurrence, because loss of cell-to-cell adhesion is a part of development of relapse tumours and that the loss of CLDN7 expression may be a specific event that is not common to all Claudins.

MME is a type II integral zinc-dependent membrane protein with neutral endopeptidase activity. The function of MME is thought to involve hydrolysis of polypeptides such as inflammatory mediators in the extracellular milieu. It regulates biological activities of peptide substrates by reducing the local concentrations available for signal transduction and receptor binding. Beside hematopoietic tissue various benign and malignant nonhaematopoietic tissues express MME (Metzgar 1981, Sato 1996). CD10 may also play a role in perpetuation of homeostasis, neoplastic transformation and tumour progression (Papandreou 1998). The experiments of the inventors show that MME is highly expressed in fibroblast-like stromal cells and in macrophage-like cells both in primary tumour and relapse tumour. Thus, MME expression in neoplastic cells could have a role in apoptosis and, above all, in proliferation, while the expression in intratumoral stromal cells may also subscribe to tumour progression (Iwaya K, Ogawa H, Izumi M, Kuroda M, Mukai K. Stromal expression of CD10 in invasive breast carcinoma: a new predictor of clinical outcome. Virchows Arch. 2002 June; 440(6):589-93. Epub 2002 Apr. 13). The exact role of MME is currently not known, but recent studies suggest that MME expression in malignant cells significantly correlates with higher apoptotic index. In the context of the present invention, the inventors could detect MME fibroblast-like stromal cells and in macrophage-like cells in GCT for the first time.

Ephrin molecules are overexpressed in a wide range of neoplasms and additional evidence suggests that overexpression is an important component of carcinogenic processes. The Ephrin receptors are divided into two subclasses: subclass A and subclass B depended on the assembly of ephrins they are binding. All Ephrin receptors of the A subclass can bind Ephrin A1. Ephrin A2 receptor binds Ephrin A1 with the highest affinity. The Ephrin and EFNA1 genes have well-established potential as classical oncogenes (Maru Y, H. H., Takaku F. 1990. Overexpression confers an oncogenic potential upon the eph gene. Oncogene, 5: 445-447). They were found as immediate early response genes of endothelium induced by TNF-α that possesses angiogenic and endothelial cell chemoattractant activity (Holzmann L B, M. R., Dixit V M. 1990. A novel immediate-early response gene of endothelium is induced by cytokines and encodes a secreted protein. Mol. Cell. Biol., 10(5830), Pandey A, S. H., Marks R M, Polyerini P J, Dixit V M. 1995. Role of B61, the ligand for the Eck receptor tyrosine kinase, in TNF-alpha-induced angiogenesis. Science, 268: 567-569). The present invention represents the expression of EFNA1 in GCT for the first time. Thus, the Ephrin-pathway may play a potential role in the progression of GCT. As such, the Ephrin family represent promising targets for diagnosis and chemotherapeutic drug development.

FGF receptors are activated by FGF ligands; the binding leads to receptor dimerisation and autophosphorylation of tyrosine residues in the cytoplasmatic domain. This stimulates intrinsic tyrosine kinase activity. The combination provides a mechanism to both recruit and phosphorylate molecules that transmit FGFR3 signals. The pathway downstream of the receptor are not well defined; implicated in the pathway are MAP (mitogen-activated protein) kinase, STAT1 (signal transduction and activation of transcription proteins) and PLCγ (phospholipase C). FGFR3 have been associated with craniosynostosais syndromes, hypochondroplasia, achondroplasia and thanatophoric dysplasia. The effect of FGFR3 signalling on bone growth is inhibitory because it is expressed in proliferating chondrocytes (Naski M C, Ornitz D M. FGF signaling in skeletal development. Front Biosci. 1998 Aug. 1; 3:D781-94.). Both in vivo and in vitro the inventors could detect FGFR3 in GCT. It is therefore contemplated that FGFR3 and its signalling pathway may play a role in progression of GCT.

Autocrine motility factor (AMF) is a tumour secreted cytokine that regulates cell motility and metastasis (Liotta, L. A., Mandler, R., Murano, G., Katz, D. A., Gordon, R. K., Chiang, P. K., & Schiffmann, E. 1986. Tumor cell autocrine motility factor. Proc Natl Acad Sci USA, 83(10): 3302-3306). AMF stimulates arbitrary and direct cell motility via its receptor AMFR. It has been shown, that increased expression of AMFR correlates with a high incidence of recurrence and a shortened survival in patients with bladder cancer and other cancer forms (Hirono, Y., Fushida, S., Yonemura, Y., Yamamoto, H., Watanabe, H., & Raz, A. 1996. Expression of autocrine motility factor receptor correlates with disease progression in human gastric cancer. Br J Cancer, 74(12): 2003-2007). Our study shows, that AMFR was only found in macrophage-like cells, mainly in foam cells. We found a significant lower expression of AMFR immunohistochemically in relapse tumours compared to primary tumours. Two relapse cases of GCT show immunohistochemically a AMFR staining of macrophage-like cells. This based on the fact, that in relapse tumour no foam cells could be detect. Therefore we describe, that the up-regulation of AMFR in primary tumour is a modified up-regulation, because the specific cell type of foam cells are lost in relapse tumours. On the basis of foam cells we could approve the complexity and heterogeneity of the GCT tissue.

Giant cell tumours of bone are local osteolytic neoplasms with variable degree of aggressiveness and a significant tendency to recur locally. Few prognostic markers have been reported to predict the clinical outcome of GCT patients, so is very important to find the factor that can be implicated in its potential aggressiveness. With cDNA microarray analysis the inventors could display new genes, in particular cdw52, efna1, fgfr3, mme, amfr and cldn7 on GCT for the first time. These genes are contemplated to be new prognostic markers which allow to differentiate between primary tumours and relapse tumours. Furthermore, the products of the above genes could be developed into targets for a selective and specific chemotherapeutical approach. First respective results as presented herein are promising.

In summary the inventors have identified a large set of genes that shed light on the molecular basis underlying the pathogenesis and progression of GCT. More studies are needed to identify the particular mechanisms that are involved in GCT. Such studies should, for example, help to determine how genes like Ephrin A1 are most effectively to be used as a marker in the diagnosis of primary and/or relapse GCT. Further characterisation of these genes/expressed sequence tags will refine the understanding of GCT and enhance the ability to diagnose and manage these patients appropriately. As the person of skill will be aware, all these approaches are encompassed by the scope of the present invention, and can easily be achieved based on the information as provided herein.

Furthermore, in particular the finding of the present invention that CDw52 is expressed in certain bone-related tumours, such as osteosarcomas and giant cell tumours, will form the basis for the extension of an anti-cancer therapy based on ligands of CDw52 and in particular of CDw52-specific antibodies such as alemtuzumab (Campath-1H). The major advantages of this novel therapy are its application in areas of the body where a surgical intervention is impossible, in the case of a multifocal disease status and the application in slowly proliferating cancers (such as giant cell tumours) where the current chemotherapeutical approaches can not be employed.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan, in this case an anti-cancer therapy based on ligands of CDw52 and other ligands as presented herein.

TREATMENT or THERAPY shall mean a process of the reversing or ameliorating or reducing a condition of a certain type or certain types of cells or tissues with respect to an abnormal behaviour, such as a proliferative disease. Preferably, a treatment is applied to a patient.

A “fragment” of a ligand, in particular a fragment of an antibody, shall mean a moiety that is derived from the ligand that is still capable of binding to the respective cellular marker (for example, CDw52). Particular examples for antibodies are scFV-fragments and other antibody-derived peptides that can bind to the respective cellular marker. In a preferred embodiment, the binding of the fragment leads to the same biological effect(s) as the binding of the full-length (or sized) ligand.

In the context of the present invention, the term “osteosarcoma” encompasses both osteosarcomas and chondrosarcomas, i.e. these terms can be used interchangeably throughout the present invention.

The present invention shall now be further described based on the following examples without being limited thereto with reference to the accompanying figures, in which

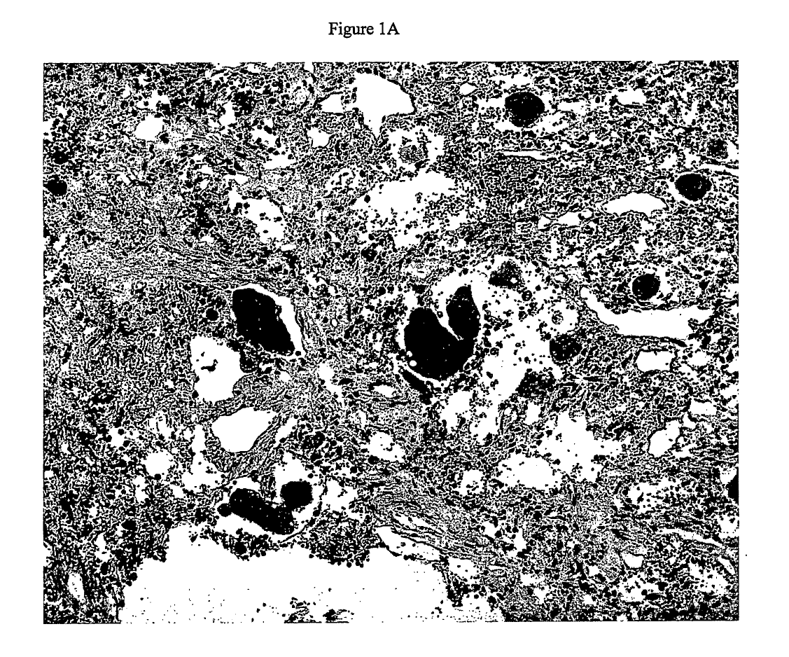

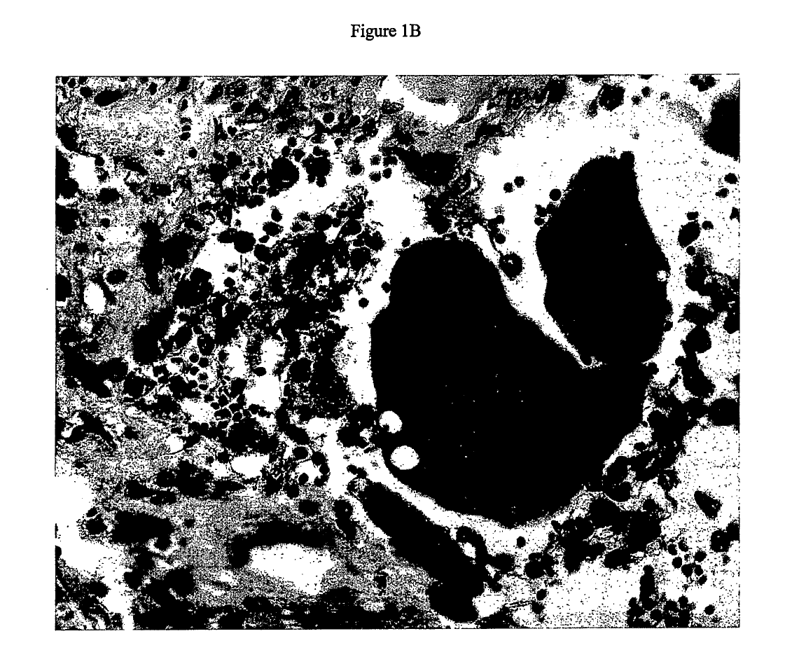

FIG. 1 shows the immunohistochemical analysis of the CDw52 expression a in giant cell tumours, and

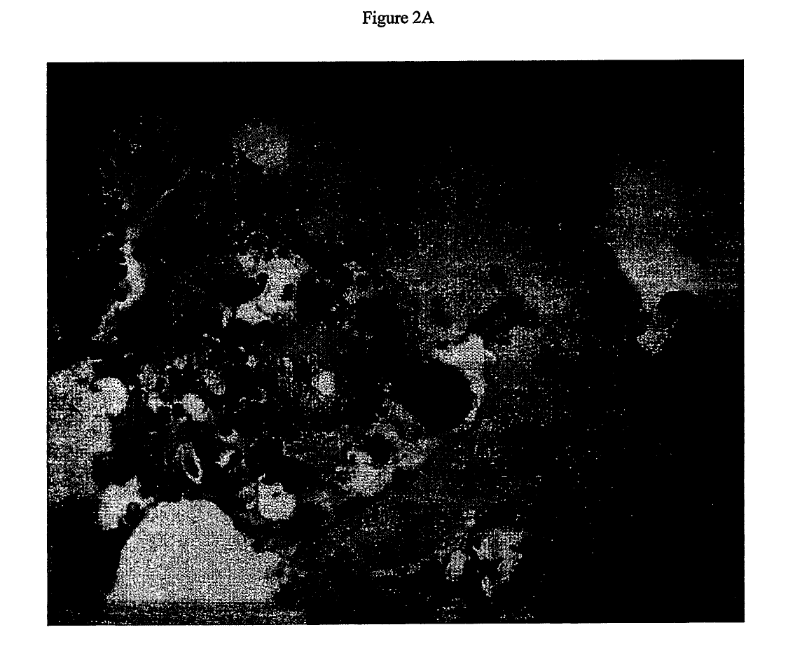

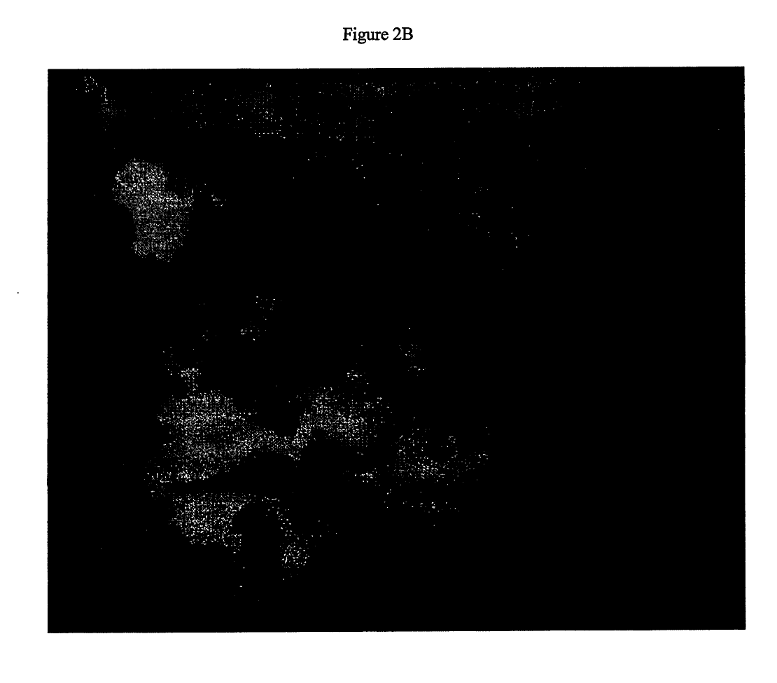

FIG. 2 shows the immunohistochemical analysis of the CDw52 expression in an osteosarcoma

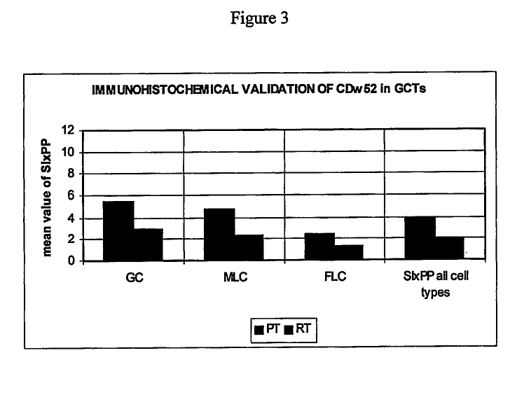

FIG. 3 shows the validation of immunohistochemical staining of CDw52 in GCTs (primary [PT] and relapse tumours [RT]) for each cell type (multinuclear giant cells [GC], macrophage-like cells [MLC] and fibroblast-like cells [FLC]) based on mean value of immunoreactivity score (SixPP, staining intensity times positively stained cells)

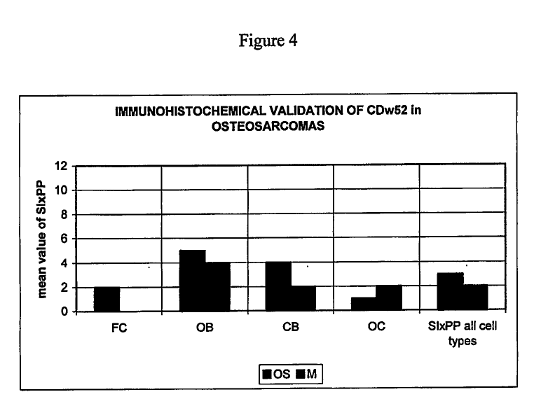

FIG. 4 shows the immunohistochemical staining of CDw52 in Osteosarcomas (OS) and lung metastasis (M) for each cell type (fibroblasts [FC], osteoblasts [OB], chondroblasts [CB] and based on mean value of immunoreactivity score (SixPP, staining intensity times positively stained cells)

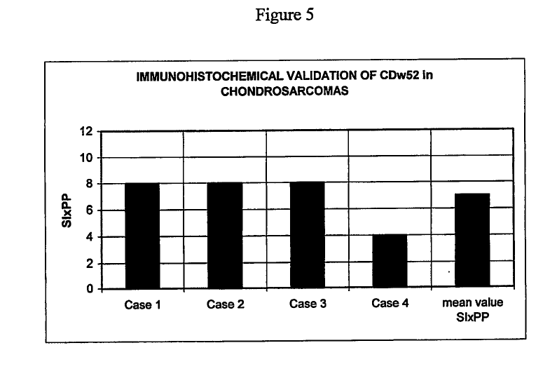

FIG. 5 shows the immunohistochemical staining of CDw52 in 4 Chondrosarcomas based on the immunoreactivity score (SixPP, staining intensity times positively stained cells)

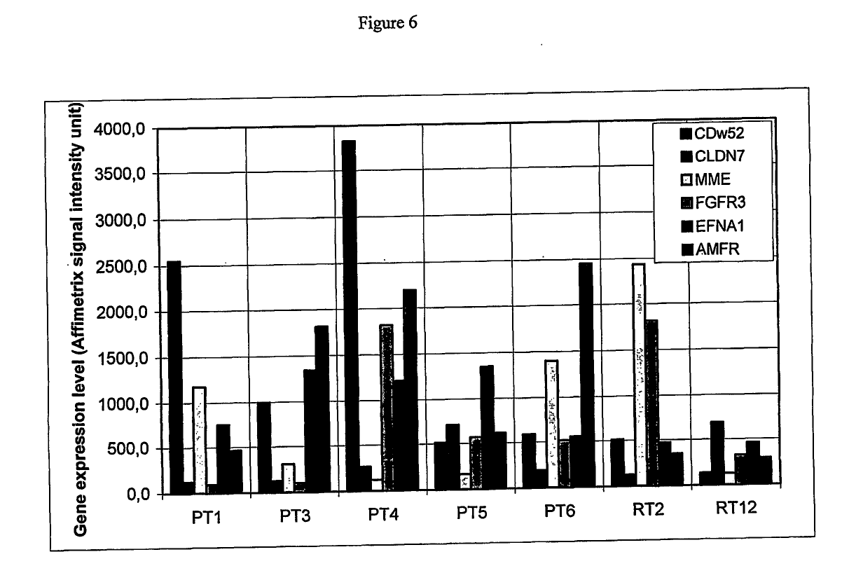

FIG. 6 shows the normalised signal intensities of 6 selected genes (mme, cdw52, efna1, cldn7, amfr, fgfr3) in 5 primary and 2 relapse GCTs obtained from microarray analysis. PT1, PT3, PT4, PT5 and PT6 are primary tumours; RT2 and RT12 are relapse tumours

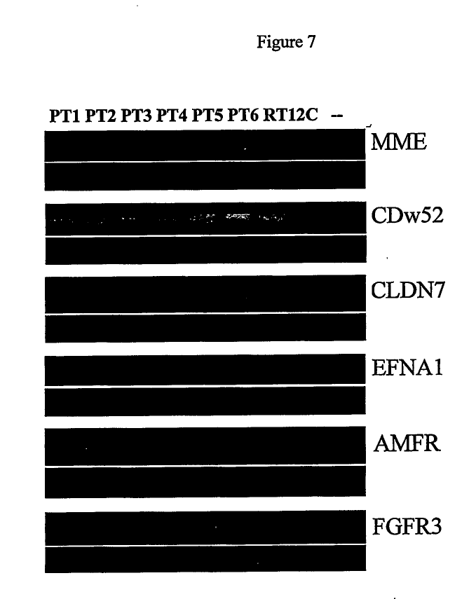

FIG. 7 shows the RT-PCR analysis of 6 selected genes in GCT. 13-Actin serves as a RNA control (lower line). RT1, RT3, RT4, RT5 and RT6 are primary tumours; RT2 and RT12 are relapse tumours, C=C-433 GCT cell line, −negative control

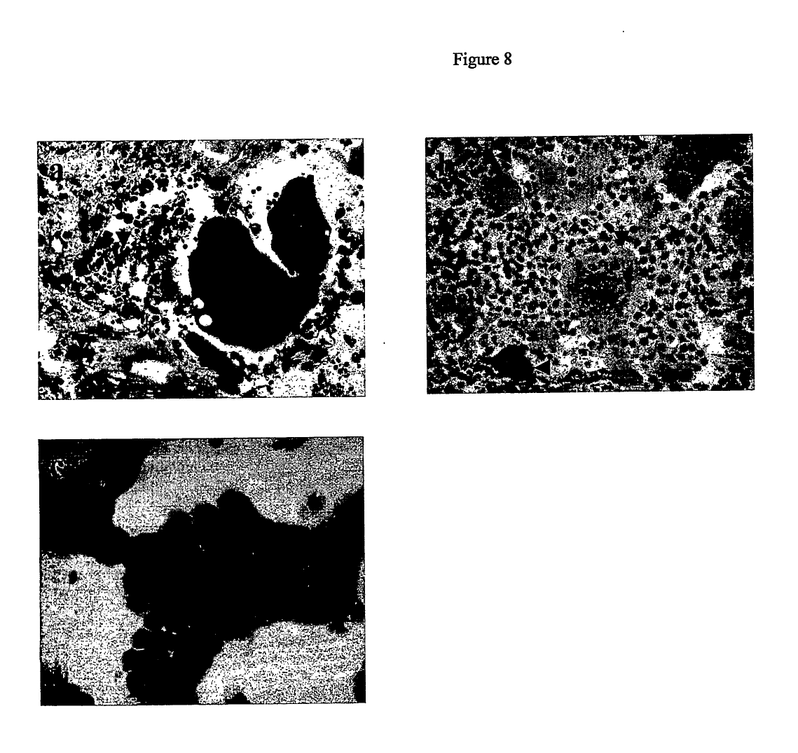

FIG. 8a-c: Immunohistochemical validation of CDw52 on GCTs. a: primary tumour, b: relapse tumour, c: GCT cell line C433; 400-times magnified.

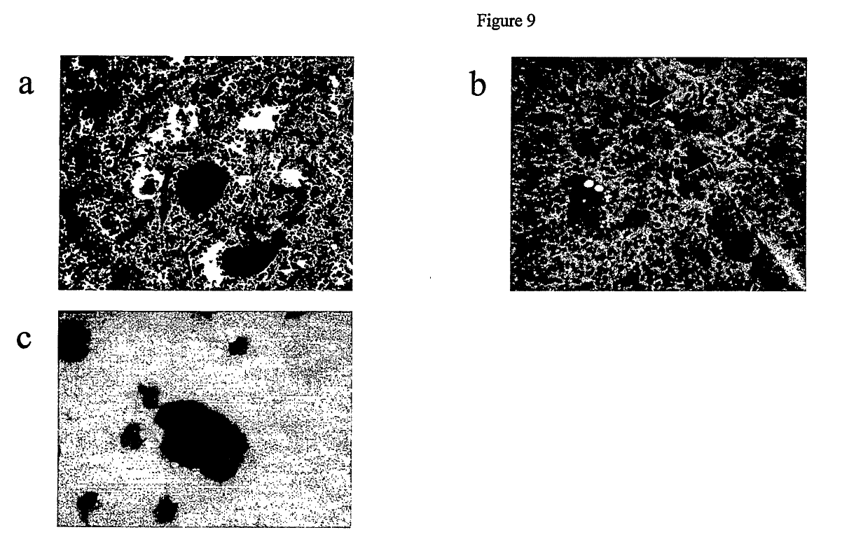

FIG. 9a-c: Immunohistochemical validation of CLDN7 on GCTs. a: primary tumour, b: relapse tumour, c: GCT cell line C-433. Negative giant cells displayed as black arrows. 400-times magnified.

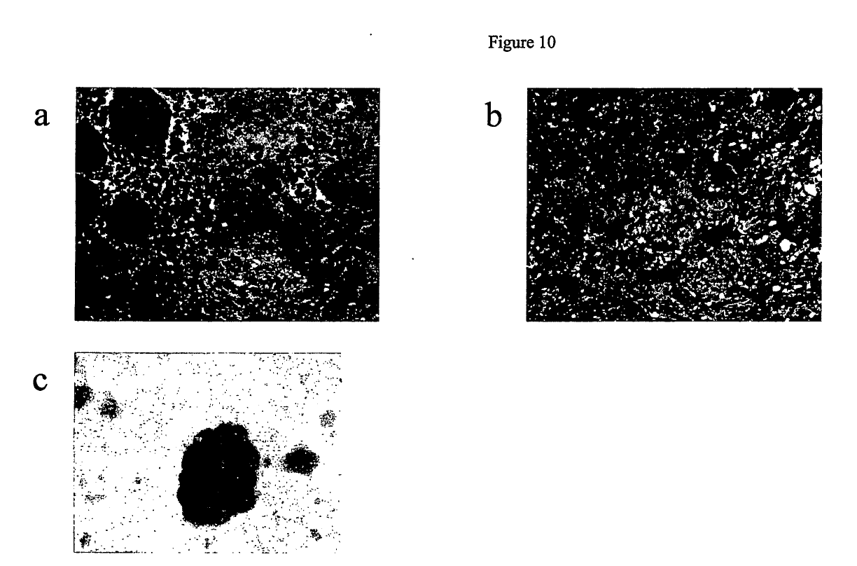

FIG. 10 a-c: Immunohistochemical validation of EFNA1 on GCTs. a: primary tumour, b: relapse tumour, c: GCT cell line C433. Negative giant cells displayed as black arrows. 400-times magnified.

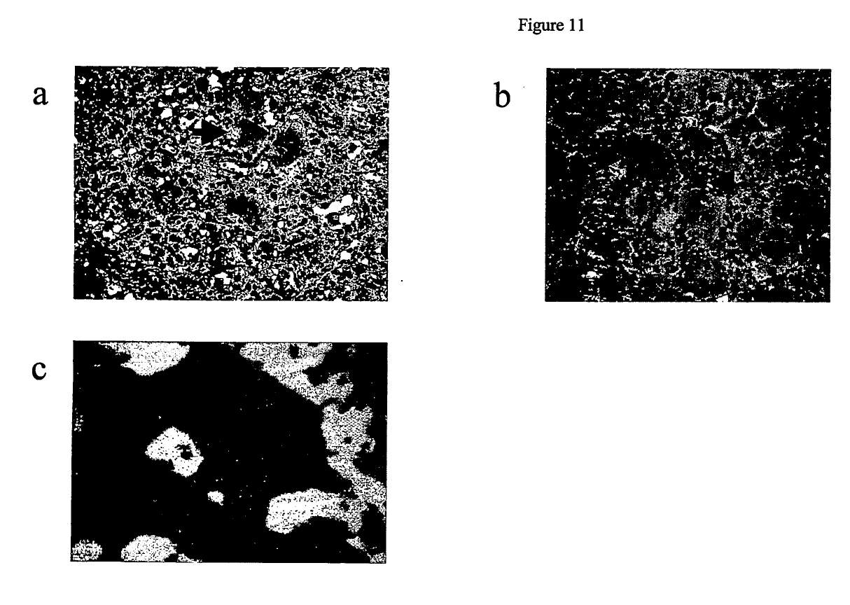

FIG. 11 a-c: Immunohistochemical validation of AMFR on GCTs. a: primary tumour, b: relapse tumour, c: GCT cell line C-433. Negative giant cells displayed as black arrows. Picture b shows single positive mononuclear macrophage-like cells (blue arrow). 400-times magnified.

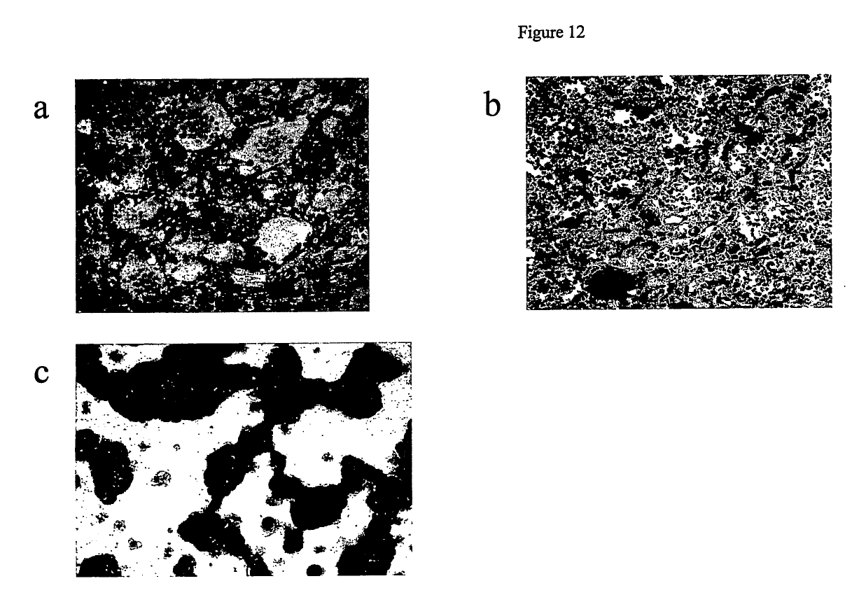

FIG. 12 a-c: Immunohistochemical validation of MME and FGFR3 on GCTs. a: MME, b: FGFR3, c: FGFR3 on GCT cell line C-433. 400-times magnified

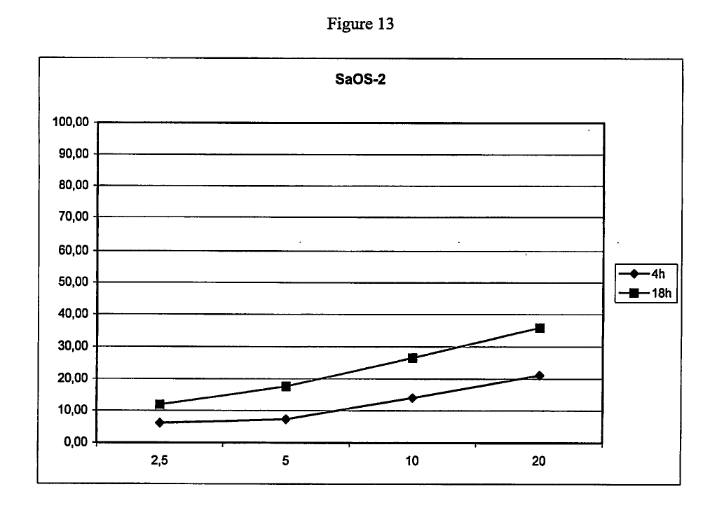

FIG. 13 shows the results of the ⁵¹Cr release assay for the osteosarcoma-cell line SaOS-2. The lysis is at approximately 45% after 18 h.

### EXAMPLES

Patients and Tissues

The GCT, OS and CS tissues were obtained from the Institute of Pathology and the Department of Trauma and Reconstructive Surgery, University Hospital Charité, Berlin, Germany, the Max-Delbrück-Center for Molecular Medicine, division of pathology, Campus Berlin Buch, Robert-Rössle-Clinical Center, Germany and from the Institute of Pathology, University of Cologne, Cologne, Germany. Primary tumours were in sano leached with tumour free margins and histologically confirmed. The GCT cell line C-433 and the OS cell line SaOS-2 were received from DSMZ (German Collection of Microorganisms and Cell Cultures) in Braunschweig, Germany.

All tissues were frozen in liquid nitrogen immediately after removal, and stored at −80° C. RNA was prepared from 7 frozen GCT samples (primary tumour n=5, relapses n=2) and used for the microarray analysis and can be used for reverse transcription-polymerase chain reaction (RT-PCR). Paraffin-embedded samples were used for immuno-histochemical analysis (see Table 1; GCT: primary tumour n=16, relapse tumour n=6, OS: tumour n=4, metastasis n=5; CS n=4).

The GCT samples (Table 1) analysed for gene expression included four men and three women with a mean age of 48.1 years (range, 24 to 70 years). Localisations of tumours were femur (n=2), pelvis (n=2), radius (n=2) and vertebral column (n=1). In the two cases of relapse tumour, complete resection of the primary tumours with tumour free margins had been confirmed histopathologically. Relapse tumours and in general primary GCTs are rare lesions, and therefore only 2 relapses and 5 primary tumour samples could be obtained for microarray study. Immunohistochemical analysis was performed on 16 primary tumours and 6 relapse GCTs on paraffin-embedded microsections. Main localisations of the GCTs were femur and tibia. For each case, hematoxylin and eosin (H&E)-stained slides were carefully reviewed and the diagnosis of primary or recurrent GCT was confirmed.

The OS samples (Table 1) analysed for CDw52 immunohistochemical expression included 6 men and 3 woman with a mean age of 42.4 years (range, 17 to 71 years). Localisations of tumours were lung in cases of metastasis (n=5) and tibia (n=2) as well as femur (n=2) in cases of OS. For each case, hematoxylin and eosin (H&E)-stained slides were stained.

The CS samples (Table 1) analysed for CDw52 immunohistochemical expression included 4 men with a mean age of 54 years (range, 43 to 71 years). Localisations of tumours were shoulder (n=2) leg (n=1) and pelvis (n=1). For each case, hematoxylin and eosin (H&E) stained slides were stained.

TABLE 1
Patient data GCTs, OS and CS

| No. | Sex | Age | Diagnosis | Localisation | IHC | Array |
|---|---|---|---|---|---|---|
| 1 | m | 53 | GCT | Femur | X | X |
| 2 | m | 53 | GCT relapse | vertebral | X | X |
| | | | | column | | |
| 3 | w | 24 | GCT | Femur | X | X |
| 4 | m | 62 | GCT | Pelvis | X | X |
| 5 | f | 28 | GCT | Radius | X | X |
| 6 | m | 70 | GCT | Pelvis | X | X |
| 7 | f | 65 | GCT | Knee | X | |
| 8 | f | 48 | GCT | Hand | X | |
| 9 | f | 47 | GCT relapse | Radius | X | |
| 10 | m | 41 | GCT | Radius | X | |
| 11 | f | 41 | GCT | Radius | X | |
| 12 | m | 67 | GCT | Trochlea | X | X |
| 13 | f | 40 | GCT | Humerus | X | |
| 14 | f | 40 | GCT | Humerus | X | |
| 15 | f | 69 | GCT | Femur | X | |
| 16 | m | 35 | GCT | Femur | X | |
| 17 | m | 44 | GCT relapse | Tibia | X | |
| 18 | m | 44 | GCT relapse | Tibia | X | |
| 19 | m | 29 | GCT relapse | Lung | X | |
| 20 | m | 31 | GCT | Femur | X | |
| 21 | m | 31 | GCT | Femur | X | |
| 22 | m | 51 | GCT relapse | Tibia | X | |
| 1 | m | 41 | OS metastasis | Lung | X | |
| 2 | w | 23 | OS metastasis | Lung | X | |
| 3 | w | 23 | OS metastasis | Lung | X | |
| 4 | m | 27 | OS | Tibia | X | |
| 5 | w | 50 | OS metastasis | Lung | X | |
| 6 | m | 71 | OS metastasis | Lung | X | |
| 7 | m | 63 | OS | Tibia | X | |
| 8 | m | 17 | OS | Femur | X | |
| 9 | m | 67 | OS | Femur | X | |
| 1 | m | 51 | CS | Shoulder | X | |
| 2 | m | 51 | CS | Shoulder | X | |
| 3 | m | 71 | CS | Leg | X | |
| 4 | m | 43 | CS | Pelvis | X | |

M = man, f = female, X at ICH = immunohistochemical staining were performed, X at array = array from these samples were performed

RNA Isolation and RT-PCR Analysis

RNA was isolated from frozen samples using the RNeasy-mini-kit (Qiagen). For RT-PCR a one-step RT-PCR kit (Cat. No. 210210) from Qiagen was used. All primer sequences and the annealing temperatures for RT-PCR for each of the genes are depicted in Table 2. β-Actin was amplified together with each RT-PCR reaction to ensure cDNA integrity.

TABLE 2
Primer sequences and annealing temperatures for 6 selected genes

| | | | Annealing |
|---|---|---|---|
| Gene | | | temper- |
| name | Forward primer | Reverse primer | ature |
| cdw52 | GCCACGAAGATCCT | GCTTGGCCCCTACAT | 52° C. |
| | ACCAAA (Seq ID | CATTA (Seq ID | |
| | No. 1) | No. 2) | |
| cldn7 | CTTGGTAGCTTGCTC | TATACATGGAGTGCA | 52° C. |
| | CTGGTAT (Seq ID | GGGACAG (Seq ID | |
| | No. 3) | No. 4) | |
| efna1 | AGGTACTGCATTCTC | TTTTTAGCAGGGACT | 56° C. |
| | TCCCATC (Seq ID | CAATGGT (Seq ID | |
| | No. 5) | No. 6) | |
| fgfr3 | TAGTTGGAGGTGATT | AGGGCCCAGTAACA | 52° C. |
| | CCAGTGA (Seq ID | GTACAGAA (Seq ID | |
| | No. 7) | No. 8) | |
| mme | CTATCCCTCACACAT | CCTACAATCCTTTCC | 54° C. |
| | CCAGACA (Seq ID | ATCTTGC (Seq ID | |
| | No. 9) | No. 10) | |
| amfr | TCGCTTAAACCAACA | AAGTTTCTCCCTCTT | 52° C. |
| | CAATCAC (Seq ID | CCTGGTC (Seq ID | |
| | No. 11) | No. 12) | |

Identification of New Genes on GCT by cDNA Microarray

Total RNA isolated from GCT tissue was quantified by UV-spectroscopy and its quality was controlled by analysis on a LabChip BioAnalyzer (AGILENT Technologies, Santa Clara, Calif.). cDNA was synthesised from 5 μg total RNA subsequently reverse transcription (RT) was performed at 43° C. Purification of double stranded cDNA was performed by phenol/chloroform extraction and precipitation using 5 M ammonium acetate and 100% ethanol at −20° C. for 20 minutes. The pellet was resuspended in 12 μl of RNase-free water. Synthesis of biotin-labelled cRNA was performed using the BioArray High Yield RNA Transcription kit (Enzo Diagnostics, Farmingdale, N.Y.). The amplified cRNA was purified on an affinity resin column (RNeasy, Qiagen, Hilden, Germany) and fragmented by incubation at 94° C. for 35 min. The amount of synthesised cRNA was determined by UV-spectroscopy and the distribution of cRNA fragments was controlled on a LabChip BioAnalyzer (AGILENT Technologies, Santa Clara, Calif.). The fragmented, biotin-labelled cRNA was hybridized to the HG U133A/MG U74Av2 array (Affymetrix, Santa Clara, Calif.). Following washing and staining, probe arrays were scanned twice at 3 μm resolution using the GeneChip System confocal scanner (Hewlett-Packard, Santa Clara, Calif.). (Affymetrix). Data analysis was performed using Microarray Suite Version 5.0 software and Data Mining Tool Version 2.0 software (Affymetrix).

Immunohistochemistry

For the additional examples, immunohistochemical analysis was performed for Claudin7, MME, AMFR, CDw52, FGFR3 and Ephrin A1 to validate the differential expression of these genes selected from microarray analysis and their proteins on GCT paraffin-embedded microsections. OS and CS paraffin-embedded microsections were stained only for CDw52. Sections were cut at 1 μm and deparaffinized through serial dilutions of alcohol. Antigen retrieval for Claudin 7, MME and AMFR was achieved by pressure cooking for 5 minutes in 0.01M sodium citrate buffer. Retrieval of the CDw52 antigen was performed by incubation with proteinase K (ready for use, DAKO) for 5 minutes and for FGFR3 and Ephrin A1 by incubation in trypsine (0.5%, Zymed Digest-All Kit) for 10 minutes at 37° C.

The primary antibodies for CLN7 (rabbit anti human, diluted 1:10 [Zymed]), for AMFR (rabbit anti human, diluted 1:20 [Abgent], for MME (mouse anti human, diluted 1:50 [Novocastra]), and for CDw52 (rat anti human CDw52, diluted 1:20 [Serotec]) were incubated for 30 minutes at room temperature. The antibody against FGFR3 (rabbit anti human, diluted 1:20 [Abgent]) and for EFNA1 (rabbit anti human, diluted 1:20 [Abgent]) were incubation over night at 4° C.

CLD7, FGFR3 and EFNA1 were detected using the LSAB-HRP-method (labelled streptavidin biotin+horseradish peroxidase), for MME and AMFR the LSAB-AP-method (labelled streptavidin biotin +. alkaline phosphatase) was used. CDw52 was detected by the double APAAP-method (alkaline phosphatase anti-alkaline phosphatase), followed by a 15 minute incubation with the biotinylated link antibody and a 15 minute incubation of the streptavidin-AP/HRP (Dako-Kit). Fuchsin⁺ and DAB were used as chromogenes and nuclear counter staining was performed with haemalaun. For APAAP both the primary antibody as well as the secondary antibody (rabbit anti rat 1:40 [DAKO]) and the APAAP complex (anti rat 1:100 [DAKO]) were incubated for 30 minutes at room temperature, followed by a 10 minute incubation of the secondary antibody and the APAAP complex.

All tumours were semi-quantitatively scored using the following scoring method modified according to Remmele and Stegner 1987: 1. A staining intensity score (SI) was established to measure the percentage of positively stained cells of each cell population (multinuclear giant cells [mGC]; macrophage-like and fibroblast-like): 0=no staining, 1=1-25%, 2=26-50%, 3=51-75%, 4=76-100%. 2. An intensity score of positively stained cells (PP) was performed as follows: 0=no expression, 1=weak expression, 2=medium expression, 3=strong expression. The relative value was calculated through SIxPP (maximum value was 12) to analyse the differences between primary tumour and relapse tumour.

Statistical Analysis

Statistical analysis was performed using unpaired t-Test. Differences were considered significant at P<0.05.

Results

To get a more comprehensive view of the gene expression of GCT, the inventors analysed the tumours using cDNA microarrays. Difficulties of the study are the rarity of GCTs. The frequency of GCTs approximately 1 million per year, half the number of osteosarcomas (Gamberi 2004). Among all primary bone tumours GCTs represent 5%. Therefore we used only 7 samples of GCT, whereas two are relapse and 5 are primary tumours. Giant cell rich tissue (giant cells of foreign body type) from periprosthetic membranes of loosened endoprostheses (n=2) served as control tissue.

Using microarrays (Affimetrix human genome U133A chip) harbouring 15.000 oligonucleotides specific for genes and expressed sequence tags, we performed a gene expression profiling of 2 non-tumorous, 5 primary (PT) and 2 relapse (RT) GCT samples. A total of 66 genes were scored as significantly differentially expressed (Table 3). Cdw52 was down-regulated in four primary GCTs as compared to the non-tumorous tissues, however not included PT4. Further down-regulation was observed in the two relapse samples as compared to the primary tumours. Cldn7 was up-regulated in one relapse tumour when compared to primary samples PT1, PT3, PT4 and PT6, whereas cldn7 was down-regulated when compared non-tumorous control tissue and primary tumour. 64 genes were detected differentially expressed upon comparison of the 5 primary tumours and the two relapse samples. Twenty two genes were detected up-regulated and 42 genes were significantly down-regulated (Table 3. FIG. 6 shows the normalised signal intensity of 6 genes for each of the primary and the relaps samples. This analysis revealed considerable heterogeneity between individual samples. Consequently most change-folds account (Cdw52 not included) are around 2, as for array evaluation we used the mean value of each group, primary tumours and relapse tumours.

In order to validate the differential expression pattern of genes in GCT found on microarray analysis, the inventors studied the same samples by RT-PCR. One upregulated gene (cldn7) and 5 downregulated genes (amfr, fgfr3, efna1, mme and cdw52) were tested and β-Actin was used as an RNA loading control (FIG. 7). The inventors also analysed the GCT cell line C-433 to demonstrate the gene expression pattern similar of GCT tissue by microarray analysis. To date neither of selected genes was known to be expressed in GCT. The inventors found that the RT-PCR analysis of the samples reflected the expression found by microarray analysis.

The inventors next investigated the expression patterns of genes in GCT at the protein level. 6 markers with available antibodies for immunohistochemical staining were studied: MME, CDw52, EFNA1, AMFR, FGFR3 and CLDN7. The expression pattern of selected genes in GCT tissue has not been reported before. Tissue from 16 primary tumours and 6 relapse tumours were used for immunohistochemical staining. GCT cell line C-433 was stained for CDw52, EFNA1, AMFR, FGFR3 and CLDN7 to compare in vivo with in vitro GCT. Additionally, positive controls for all selected genes were stained. The immunohistochemical score for each gene were listed in Table 4.

CDw52 were expressed in all three cell populations both in primary and in relapse GCT (FIGS. 3, 8A). The tissue was very heterogeneous; therefore the inventors could detect highly stained giant and macrophage like cells (FIG. 8A) just like very weak stained cells. The mean value of the hypothetical account show a medium staining in giant cells and macrophage-like cells, in the stromal-like cell population the staining was light (Table 4). A significant difference between primary tumour and relapse tumour was established only for giant cells (p=0.041) and macrophage-like cells (p=0.05), however, not for the fibroblast like cells (SIxPP 2.4 in primary tumour, SIxPP 1.3 in relapse tumour). Consequently, the GCT cell line show a very weak staining of the fibroblast-like stromal cells.

In an immunohistochemical analysis of the CDw52 expression in osteosarcoma tumours (see FIGS. 2 and 4) and chondrosarcoma (see FIG. 5), the expression of CDw52 could be verified. The level of expression is very high in CS, whereas for OS the expression is similar to GCT. CDw52 expression could be almost exclusively found in the cytoplasm of the tumour cells.

Claudin 7 showed a significantly decreased expression in all three cell types in relapse GCT tumours when compared to primary samples (FIG. 8B). Semi-quantitative evaluation of the staining intensity revealed a two times higher score in giant cells (p=0.001), three times higher in macrophage-like cells (p=0.000001) and actually 3.5 times higher in fibroblast-like cells (p=0.001) in primary tumour when compared to recurrent tumours (Table 4, FIGS. 9A, B). Interestingly, in the relapse tumours there were a number of negatively stained giant cells surrounded by positive cells (FIG. 9B black arrows). High levels of Claudin 7 protein were detected in the fibroblast-like C-433 giant cell tumour line thus, indicating a correlation between the in vivo and the in vitro observations.

Significant differences between primary tumour and relapse tumour in all three cell populations in GCT (giant cells p=0.001, macrophage-like cells p=0.003, fibroblast-like cells p=0.001) are shown for Ephrin A1 (Table 4). Giant cells display a intensive staining in primary tumour (FIGS. 8C, 9C), whereas in relapse tumour negative giant cells to be in immediate neighbor to medium positive stained giant cells (FIG. 9D). The fibroblast-like cells show in vivo a lesser staining then in the cell line (FIG. 8C).

The autocrine motility factor (AMFR) is limited to the macrophage-like cells, especially for foam cells (FIG. 8D). In relapse tumour are only two cases with AMFR-staining on macrophage-like cells were found (FIG. 9F); therefore a significant difference between primary and relapse tumour could be detected (p=0.001).

MME is restricted to macrophage- and fibroblast-like cells with an intensive immunohistochemical staining (FIG. 8E); whereas staining for FGFR3 is available in all cell populations with light staining (FIG. 8F) and in the GCT cell line C-433 with strong staining.

TABLE 3
Highly up- and downregulated genes in GCT
(primary tumour versus relapse tumour)*

| | Accession | Change |
|---|---|---|
| Gene name | number | fold |
| up | | |
| Hypothetical protein MGC4694 | BC004911.1 | 3.0 |
| Decay accelerating factor for | NM_000574.1 | 2.4 |
| complement (CD55, Cromer blood | | |
| group system) | | |
| Seizure related gene 6 (mouse)-like | AL050253.1 | 2.4 |
| Tripeptidyl peptidase II | NM_003291.1 | 2.4 |
| 7r05c01.x1 NCI_CGAP_Lu24 | BF939092 | 2.4 |
| hk42a07.y1 NCI_CGAP_Ov34 | AI254547 | 2.4 |
| qb33c06.x1 | AI144007 | 2.3 |
| Soares_pregnant_uterus_NbHPU | | |
| AU134977 PLACE1 | AU134977 | 2.3 |
| SH3-domain GRB2-like 3 | NM_003027.1 | 2.3 |
| POU domain, class 5, transcription | AF268615.1 | 2.3 |
| factor 1 | | |
| Homo sapiens guanylate cyclase | L36861 | 2.3 |
| activating protein (GCAP) | | |
| 60S Ribosomal protein L21 (RPL21) | AL356414 | 2.2 |
| pseudogene | | |
| 2,4-dienoyl CoA reductase 2, | NM_020664.1 | 2.2 |
| peroxisomal | | |
| Solute carrier family 16 | NM_004694.1 | 2.2 |
| (monocarboxylic acid transporters), | | |
| member 6 | | |
| 602407908F1 NIH_MGC_91 | BG283584 | 2.1 |
| zb44c04.s1 Soares_fetal_lung_NbHL19W | N92708 | 2.1 |
| Claudin 7 (CLDN7) | NM_001307.1 | 2.1 |
| Killer cell lectin-like receptor | U30610.1 | 2.1 |
| subfamily D, member 1 | | |
| Cytidine monophosphate-N- | AF074480.1 | 2.1 |
| acetylneuraminic acid hydroxylase | | |
| (CMP-N-acetylneuraminate monooxygenase) | | |
| AU155515 PLACE1 | AU155515 | 2.0 |
| Desmoglein 3 (pemphigus vulgaris | NM_001944.1 | 2.0 |
| antigen) | | |
| Bromodomain adjacent to zinc finger | AA788652 | 2.0 |
| domain, 2A | | |
| MHC class II transactivator | U18288.1 | 2.0 |
| down | | |
| CDW52 antigen (CAMPATH-1 antigen) | NM_001803.1 | 25.8 |
| T cell receptor gamma locus | M30894.1 | 5.2 |
| T cell receptor gamma locus | M16768.1 | 4.0 |
| Autocrine motility factor receptor | NM_001144.1 | 3.7 |
| Hypothetical protein FLJ22316 | NM_025080.1 | 3.3 |
| MAGE-E1 protein | NM_030801.1 | 3.1 |
| Hypothetical protein FLJ20399 | NM_017803.1 | 2.7 |
| Hypothetical protein FLJ10628 | NM_018159.1 | 2.6 |
| SRY (sex determining region Y)-box 9 | NM_000346.1 | 2.6 |
| (campomelic dysplasia, autosomal | | |
| sex-reversal) | | |
| Membrane metallo-endopeptidase | NM_007287.1 | 2.6 |
| (neutral endopeptidase, enkepha- | | |
| linase, CALLA, CD10) | | |
| KIAA0690 protein | AK021460.1 | 2.6 |
| Hypothetical protein FLJ23191 | NM_024574.1 | 2.6 |
| T cell receptor delta locus | X06557.1 | 2.6 |
| Lymphocyte-specific protein 1 | NM_002339.1 | 2.5 |
| KIAA0401 protein | AB007861.1 | 2.5 |
| Fibroblast growth factor receptor 3 | NM_000142.2 | 2.4 |
| (achondroplasia, thanatophoric | | |
| dwarfism) | | |
| qz26f08.x1 NCI_CGAP_CLL1 | AI356398 | 2.3 |
| zj32g01.s1 | AI346341 | 2.3 |
| Soares_fetal_liver_spleen_1NFLS_S1 | | |
| Acyloxyacyl hydrolase (neutrophil) | NM_001637.1 | 2.3 |
| Dentatorubral-pallidoluysian atrophy | D31840.1 | 2.3 |
| (atrophin-1) | | |
| AU147942 MAMMA | AU147942 | 2.2 |
| BTB (POZ) domain containing 2 | NM_017797.1 | 2.2 |
| SAR1 protein | BC003658.1 | 2.2 |
| xd88h01.x1 Soares_NFL_T_GBC_S1 | AW117368 | 2.2 |
| H. sapiens HEK2 mRNA for protein | D16845.1 | 2.2 |
| tyrosine kinase receptor. | | |
| ze75h04.s1 Soares_fetal_heart_NbHH19W | AA053967 | 2.1 |
| Lymphocyte antigen 6 complex, locus E | NM_002346.1 | 2.1 |
| Nuclear factor of activated T-cells, | NM_004554.1 | 2.1 |
| cytoplasmic, calcineurin-dependent 4 | | |
| CGI-96 protein | NM_015703.1 | 2.1 |
| Leukocyte immunoglobulin-like receptor, | AF009634.1 | 2.1 |
| subfamily B member 3 | | |
| WW domain-containing oxidoreductase | NM_018560.2 | 2.1 |
| Tumour suppressing subtransferable | NM_005706.1 | 2.1 |
| candidate 4 | | |
| Potassium voltage-gated channel, | NM_000218.1 | 2.1 |
| KQT-like subfamily, member 1 | | |
| Cubilin (intrinsic factor-cobalamin | NM_001081.1 | 2.1 |
| receptor) | | |
| Ephrin-A1 | NM_004428.1 | 2.1 |
| ELK1 pseudogene (ELK2) and immuno- | AB016195 | 2.1 |
| globulin heavy chain gamma pseudogene | | |
| (IGHGP) | | |
| Hypothetical protein FLJ20699 | NM_017931.1 | 2.1 |
| Stathmin 1/oncoprotein 18 | NM_005563.2 | 2.0 |
| Hypothetical protein dJ462O23.2 | NM_020448.1 | 2.0 |
| Tubulin-specific chaperone d | BC006364.1 | 2.0 |
| 602136853F1 NIH_MGC_83 | BF674712 | 2.0 |
| Interleukin 10 | NM_000572.1 | 2.0 |

Shown are all genes with differential expression between primary tumour and relapse tumour with a change fold of at least 2.
*CDw52 was down-regulated when compared non-tumorous-tissue and GCT experiment (primary tumours and relapse tumours), CLDN7 was up-regulated when compared non-tumorous-tissue vs. primary tumour vs. relapse tumour.

TABLE 4
Semi-quantitative scoring results of 6 selected genes for multinuclear giant cells
(mGC), macrophage-like and fibroblast-like mononuclear cells. P < 0.05

| | mGC | | | | | | | | | Macrophage-like cells | | | | |
|---|---|---|---|---|---|---|---|---|---|---|---|---|---|---|
| Gene | PT | | | | Rez | | | | | PT | | | | Rez |
| name | % | PP | SI | Hyp | % | PP | SI | Hyp | P | % | PP | SI | Hyp | % |
| CDw52 | 16/16 | 2.9 | 1.9 | 5.5* | 5/6 | 1.7 | 1.3 | 3.0 | 0.041 | 16/16 | 2.9 | 1.6 | 4.8* | 4/6 |
| | (100) | | | | (83.3) | | | | | (100) | | | | (66.7) |
| Claudin | 16/16 | 3.8 | 2.6 | 10.2* | 6/6 | 3.0 | 1.7 | 5.0 | 0.001 | 16/16 | 2.3 | 2.3 | 7.1* | 6/6 |
| 7 | (100) | | | | (100) | | | | | (100) | | | | (100) |
| AMFR | 0/16 | 0.0 | 0.0 | 0.0 | 0/6 | 0.0 | 0.0 | 0.0 | / | 16/16 | 1.3 | 2.3 | 2.9* | 2/6 |
| | (0) | | | | (0) | | | | | (100) | | | | (33.3) |
| Ephrin | 16/16 | 3.4 | 2.7 | 9.2* | 6/6 | 2.3 | 1.8 | 4.5 | 0.001 | 16/16 | 2.4 | 2.0 | 4.9* | 4/6 |
| A1 | (100) | | | | (100) | | | | | (100) | | | | (66.7) |
| MME | 0/16 | 0.0 | 0.0 | 0.0 | 0/6 | 0.0 | 0.0 | 0.0 | / | 16/16 | 2.8 | 2.8 | 8.3 | 6/6 |
| | (0) | | | | (0) | | | | | (100) | | | | (100) |
| FGFR3 | 12/16 | 1.4 | 1.1 | 2.4 | 6/6 | 2.0 | 1.7 | 3.5 | 0.432 | 12/16 | 2.7 | 1.1 | 2.2 | 5/6 |
| | (75) | | | | (100) | | | | | (75) | | | | (83.3) |

| | |
|---|---|
| Macrophage- | |
| like cells | Fibroblast-like cells |

| Gene | Rez | | | | PT | | | | Rez | | | | |
|---|---|---|---|---|---|---|---|---|---|---|---|---|---|
| name | PP | SI | Hyp | P | % | PP | SI | Hyp | % | PP | SI | Hyp | P |
| CDw52 | 1.3 | 1.0 | 2.3 | 0.05 | 15/16 | 1.8 | 1.3 | 2.4 | 4/6 | 1.3 | 0.7 | 1.3 | 0.227 |
| | | | | | (93.8) | | | | (66.7) | | | | |
| Claudin | 1.8 | 1.2 | 2.2 | 1E−5 | 16/16 | 2.8 | 1.9 | 5.8* | 6/6 | 1.3 | 1.2 | 1.7 | 0.001 |
| 7 | | | | | (100) | | | | (100) | | | | |
| AMFR | 0.3 | 0.5 | 0.5 | 0.001 | 0/16 | 0.0 | 0.0 | 0.0 | 0/6 | 0.0 | 0.0 | 0.0 | / |
| | | | | | (0) | | | | (0) | | | | |
| Ephrin | 1.7 | 1.8 | 1.3 | 0.003 | 16/16 | 2.0 | 1.6 | 3.6* | 1/6 | 0.2 | 0.3 | 0.3 | 0.001 |
| A1 | | | | | (100) | | | | (16.7) | | | | |
| MME | 3.0 | 3.0 | 7.7 | 0.782 | 16/16 | 3.0 | 2.8 | 8.4 | 6/6 | 3.0 | 2.7 | 8.5 | 0.946 |
| | | | | | (100) | | | | (100) | | | | |
| FGFR3 | 1.5 | 2.0 | 1.7 | 0.567 | 14/16 | 1.8 | 1.7 | 3.5 | 5/6 | 1.7 | 2.7 | 3.2 | 0.798 |
| | | | | | (87.5) | | | | (83.3) | | | | |

(%) = percentage of positive cases; SI = mean value of positive staining score; PP = mean value of staining intensity score; Hyp = immunoreactivity score (SI × PP); P = significant differences between primary tumours compared with relapse tumours; PT = primary tumour, Rez = relapse tumour.

Antibody-Mediated Cellular Cytotoxicity of CD52 (ADCC, ⁵¹Cr Release Assay)

A ⁵¹Cr release assay was performed with antibodies of CD52 using commonly available SaOS-2 cells as osteosarcoma-cell line. For the assay, the following protocol (according to Rebello P and Hale G 2002, slightly modified) was used.
Draw 30 ml blood from the patient to be analysed
Ficoll-resolution of the LAK-cells by gradient-centrifugation
30 ml blood+30 ml 1×PBS
15 ml Ficoll-solution in 50 ml tubes; add PBS/blood
centrifuge 10 min 500×g
remove lymphocytes (LAK-cells) using pipette (turbid phase) and transfer into new tube
wash 2×5 min 1000 rpm (1 ml with 1×PBS, 1× with medium)
count cells
add 2000 IU/10 ml IL-2
incubate 72 h at 37° C.
determine cell-count of the cells to be examined
Centrifuge 5 min 1000 rpm at 4° C.
resuspend cells in 10 ml medium
1*10⁶ cells/1.5 ml; label with 0.5 mCi ⁵¹Cr
incubate 2 h 37° C. (shake every 30 min)
prepare PAK 1:100 (sterile)
prepare LAK-cells (cells were counted and diluted to concentrations 1:2.5-1:5 ect.)
fill plates with all samples (200 μl per well, 100 μl Target-cells, 75 μl LAK-cells (only at E:T-ratio), 25 μl CAMPATH, 10 μl SDS (at maximum release), add to 200 μl)
Centrifuge plate at 1000 rpm 10 min
put 80 μl of supernatant into tubes
measure ⁵¹Cr in supernatant
after 18 h, centrifuge again at 1000 rpm for 10 min
put 80 μl of supernatant into tubes
measure ⁵¹Cr in supernatant

FIG. 13 shows the results of the assay for the osteosarcoma-cell line SaOS-2. The lysis is at approximately 45% after 18 h.

Treatment of a Solid Tumour Based on a CDw52 Specific Antibody

Treatment Scheme A):

The patients (optionally including chemotherapy-naive patients) will receive 3, 10, and 30 mg of Campath-1H on sequential days, followed by 30 mg three times weekly, as 2-hour intravenous infusions, for 4 to 12 weeks. The objective response rate will be expected at about 50% (40% to 63%), with an about 40% complete response (CR) rate (28% to 51%). The most common Campath-1H-related adverse events will be expected as acute reactions during or immediately after infusions. Infectious episodes during treatment may lead to treatment discontinuation.

Treatment Scheme B):

As intravenous Campath-1H is almost invariably associated with reactions, sometimes of WHO grade 3-4, the subcutaneous route of administration will be adopted, which proves to induce rare and mild adverse reactions but usually has comparable efficacy.

The patients who, optionally, responded to FAMP will received subcutaneous Campath-1H, three times a week for 6 weeks in escalating doses up to 10 mg. The patients will receive acyclovir and cotrimoxazole as infection prophylaxis. Granulocyte colony-stimulating factor (GCSF), at the dosage of 5-10 μg/kg/day, or intermediate-dose Ara-C (800 mg/m(2)/q 12 h×6 doses), can be administered to obtain peripheral blood stem cell (PBSC) mobilisation.

Treatment Scheme C):

The patient will received treatment with CAMPATH-1H (2 mg/day iv for five days, two days rest, then 10 mg/day iv for five days). The “double-peaked” pattern will be seen with neutrophils which increases during the same time course and coincides with the second and third dosing days at 2 and 10 mg respectively (10 mg doses will follow a two day rest).

The 10 dose CAMPATH-1H treatment course should be well tolerated. Fever and headache might be reported as adverse events with both occurring with administration of the first 2 mg and 10 mg doses.

The person of skill will be readily able to amend the above given treatment schemes with respect to other ligands, such as protein, in particular protein or peptide mimetica of anti-CDw52 antibodies, glycans and P-selectin. Furthermore, the protocols can be amended in order to be applied for other antigens as described herein, such as CLDN7, MME, AMFR, EFNA1, and FGFR3.

1. A method for treating a solid tumour that expresses at least one cellular marker selected from the group comprising CDw52, Claudin 7, Ephrin A1, AMFR, MME, and FGFR3, wherein said method comprises administering to the tumour a ligand of one of said markers.

2. The method according to claim 1, wherein the cellular marker expressing solid tumour is a bone tumour.

3. The method according to claim 1, wherein the cellular marker expressing solid tumour is selected from the group consisting of giant cell tumours, chondrosarcomas, and osteosarcomas.

4. The method according to claim 1, wherein the ligand is a cellular marker-specific antibody, a fragment thereof, a cellular marker-binding peptide, or a cellular marker-interacting substance.

5. The method according to claim 1, wherein the ligand is alemtuzumab (Campath-1H).

6. The method according to claim 1, wherein the ligand is administered systemically or locally, or both.

7. The method according to claim 1, wherein the ligand is administered in a pharmaceutical composition to a patient and the ligand is present in the composition at a concentration that provides an in vivo concentration of said ligand in the patient of between 0.01 mg/kg/day and 1 mg/kg/day.

8. The method according to claim 1, wherein the ligand is administered in combination with at least one other chemotherapeutically active substance.

9. The method according to claim 1, wherein the ligand is for a specific treatment of mGCs, macrophage-like cells, and fibroblast-like cells of the tumour.

10. A method for diagnosing a solid tumour wherein said method utilizes a cellular marker selected from the group consisting of CDw52, Claudin 7, Ephrin A1, AMFR, MME, and FGFR3.

11. The method according to claim 11, wherein the cellular marker expressing solid tumours is a bone tumour.

12. The method according to claim 10, wherein the cellular marker expressing solid tumour is selected from the group consisting of giant cell tumours, chondrosarcomas, and osteosarcomas.

13. The method according to claim 10, wherein the diagnosis comprises distinguishing between mGCs, macrophage-like cells, and fibroblast-like cells of the tumour.

14. An improved method for screening for ligands of a cellular marker selected from the group consisting of CDw52, Claudin 7, Ephrin A1, AMFR, MME, and FGFR3, wherein said method comprises the steps of:
a) incubating a cell expressing at least one marker selected from CDw52, Claudin 7, Ephrin A1, AMFR, MME, and FGFR3 with a putative ligand,
b) measuring if a binding between at least one marker selected from CDw52, Claudin 7, Ephrin A1, AMFR, MME, and FGFR3 and said putative ligand occurs, and
c) in the case of a binding of said ligand to at least one marker is measured, measuring if said binding between said at least one marker and said identified ligand also leads to a marker-mediated death of a marker-expressing solid tumour cell.

15. A method for the production of a pharmaceutical formulation, comprising the steps of:
a) incubating a cell expressing at least one marker selected from CDw52, Claudin 7, Ephrin A1, AMFR, MME, and FGFR3 with a putative ligand,
b) measuring if a binding between at least one marker selected from CDw52, Claudin 7, Ephrin A1, AMFR. MME, and FGFR3 and said putative ligand occurs,
c) in the case of a binding of said ligand to at least one marker is measured, measuring if said binding between said at least one marker and said identified ligand also leads to a marker-mediated death of a marker-expressing solid tumour cell, and
d) formulating a ligand identified in steps a) through c) with at least one pharmaceutically acceptable carrier and/or excipient.
